(19) United States
(12) Patent Application Publication
(10) Pub. No.: US 2024/0071417 A1
(43) Pub. Date: Feb. 29, 2024

(54) HOLOGRAM RECORDING MEDIUM, HOLOGRAM OPTICAL ELEMENT, OPTICAL DEVICE, OPTICAL COMPONENT, AND METHOD FOR FORMING HOLOGRAM DIFFRACTION GRATING

(71) Applicant: SONY GROUP CORPORATION, TOKYO (JP)

(72) Inventors: SHOICHI OZAWA, TOKYO (JP); TAKAHIRO OHE, TOKYO (JP); HISAYA HARA, TOKYO (JP); KENSHIRO KAWASAKI, TOKYO (JP)

(21) Appl. No.: 18/260,345

(22) PCT Filed: Sep. 29, 2021
(86) PCT No.: PCT/JP2021/035926

§ 371 Date: Jul. 5, 2023

(30) Foreign Application Priority Data
Jan. 13, 2021 (JP) 2021-003831

(51) Int. Cl. G11B 7/24044; G03H 1/02; G11B 7/245

(52) U.S. Cl. CPC G11B 7/24044; G03H 1/0244; G11B 7/245; G03H 2001/0264; G03H 2260/12

(57) ABSTRACT

Provided is a hologram recording medium that can achieve suppression of deterioration of the storage stability in an unexposed state. The hologram recording medium includes a base material and a photosensitive layer. The oxygen permeability of the base material measured in an environment at 23° C. and 0% RH is more than 0.1 cm³·(m²·day·atm)⁻¹ and 10000 cm³·(m²·day·atm)⁻¹ or less. The photosensitive layer contains a polymerizable compound, and the polymerizable compound contains a compound represented by a specific general formula.

### TECHNICAL FIELD

The present disclosure relates to a hologram recording medium, a hologram optical element, an optical device including the hologram optical element, an optical component, and a method for forming a hologram diffraction grating.

### BACKGROUND ART

In a hologram recording medium, a light and dark (interference) pattern of light is recorded as a pattern of, for example, the refractive index on a photosensitive layer. Hologram recording media are widely used in fields of optical information processing, security, medicine, head-up displays, and the like. A hologram recording medium enables recording of three-dimensional information of an object as a large capacity of optical information, and therefore has attracted attention as a next-generation recording medium.

In the photosensitive layer used for information recording, a polymerization reaction is inhibited by oxygen in the air. Therefore, in order to suppress intrusion of air from the outside, characteristics of a base material (shielding material) to cover the photosensitive layer have been studied. For example, Patent Document 1 proposes a shielding material, for a hologram recording medium, having an oxygen permeability coefficient at 30° C. of 400 (cm³·100 μm/m²/24 hr/atm) or less.

### CITATION LIST

### Patent Document

### Patent Document 1: Japanese Patent No. 2011-107182

### SUMMARY OF THE INVENTION

### Problems to be Solved by the Invention

However, in a hologram recording medium in which a base material (shielding material) covering a photosensitive layer has a low oxygen permeability coefficient, the storage stability in an unexposed state deteriorates.

An object of the present disclosure is to provide a hologram recording medium in which deterioration of the storage stability in an unexposed state can be suppressed, a hologram optical element, an optical device including the hologram optical element, an optical component, and a method for forming a hologram diffraction grating.

### Solutions to Problems

In order to solve the above-described problems, a first disclosure is

a hologram recording medium including

a base material and a photosensitive layer,

the base material having an oxygen permeability of more than 0.1 cm³·(m²·day·atm)⁻¹ and 10000 cm³·(m²·day·atm)⁻¹ or less, the oxygen permeability measured in an environment at 23° C. and 0% RH, the photosensitive layer containing a polymerizable compound, the polymerizable compound containing a compound represented by a general formula (1) described below.

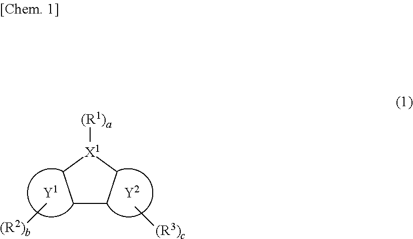

In the general formula (1), X¹ represents an oxygen atom, a nitrogen atom, a phosphorus atom, a carbon atom, or a silicon atom. In a case where X¹ represents an oxygen atom, a is 0, in a case where X¹ represents a nitrogen atom or a phosphorus atom, a is 1, and in a case where X¹ represents a carbon atom or a silicon atom, a is 2.

Y¹ and Y² each represent a benzene ring or a naphthalene ring. In a case where Y¹ and/or Y² represents a benzene ring, b or c corresponding to Y¹ and/or Y² representing the benzene ring is 4. In a case where Y¹ and/or Y² represents a naphthalene ring, b and/or c corresponding to Y¹ and/or Y² representing the naphthalene ring is 6.

R¹ to R³ each represent hydrogen or a substituent represented by *—Z¹(R⁴)_{d} (in which * represents a binding site). In a case where a plurality of R¹s, a plurality of R²s, and a plurality of R³s are present, the plurality of R¹s, the plurality of R²s, and the plurality of R³s may be of an identical kind or of different kinds, and all of the plurality of R¹s, the plurality of R²s, and the plurality of R³s in the general formula (1) are not simultaneously hydrogen.

Z¹ represents a single bond, a divalent or higher saturated hydrocarbon group, or a divalent or higher unsaturated hydrocarbon group, and the saturated hydrocarbon group or the unsaturated hydrocarbon group may contain an ether bond and/or a thioether bond. In a case where Z¹ represents a single bond, d is 1, and in a case where Z¹ represents a saturated hydrocarbon group or an unsaturated hydrocarbon group, d is an integer of 1 or more.

R⁴ represents hydrogen or a polymerizable substituent. In a case where a plurality of R⁴s is present, the plurality of R⁴s may be of an identical kind or of different kinds, and all of the plurality of R⁴s in the general formula (1) is not simultaneously hydrogen.

A second disclosure is a hologram optical element including the hologram recording medium of the first disclosure, in which

the polymerizable compound is polymerized.

A third disclosure is an optical device including the hologram optical element of the second disclosure.

A fourth disclosure is an optical component including the hologram optical element of the second disclosure.

A fifth disclosure is
a method for forming a hologram diffraction grating, the method including
selectively reacting a hologram recording medium including a base material and a photosensitive layer, by using an electromagnetic ray having an amplitude modulated spatially,
the base material having an oxygen permeability of more than 0.1 cm³·(m²·day·atm)⁻¹ and 10000 cm³·(m²·day·atm)⁻¹ or less, the oxygen permeability measured in an environment at 23° C. and 0% RH,
the photosensitive layer containing a polymerizable compound, the polymerizable compound represented by a general formula (1) described below.

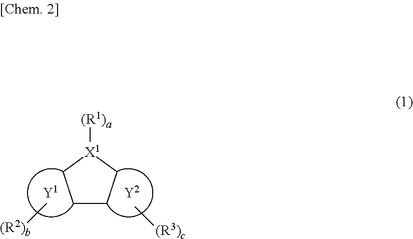

In the general formula (1), X¹ represents an oxygen atom, a nitrogen atom, a phosphorus atom, a carbon atom, or a silicon atom. In a case where X¹ represents an oxygen atom, a is 0, in a case where X¹ represents a nitrogen atom or a phosphorus atom, a is 1, and in a case where X¹ represents a carbon atom or a silicon atom, a is 2.

Y¹ and Y² each represent a benzene ring or a naphthalene ring. In a case where Y¹ and/or Y² represents a benzene ring, b or c corresponding to Y¹ and/or Y² representing the benzene ring is 4. In a case where Y¹ and/or Y² represents a naphthalene ring, b and/or c corresponding to Y¹ and/or Y² representing the naphthalene ring is 6.

R¹ to R³ each represent hydrogen or a substituent represented by *—Z¹(R⁴)_{d} (in which * represents a binding site). In a case where a plurality of R¹s, a plurality of R²s, and a plurality of R³s are present, the plurality of R¹s, the plurality of R²s, and the plurality of R³s may be of an identical kind or of different kinds, and all of the plurality of R¹s, the plurality of R²s, and the plurality of R³s in the general formula (1) are not simultaneously hydrogen.

Z¹ represents a single bond, a divalent or higher saturated hydrocarbon group, or a divalent or higher unsaturated hydrocarbon group, and the saturated hydrocarbon group or the unsaturated hydrocarbon group may contain an ether bond and/or a thioether bond. In a case where Z¹ represents a single bond, d is 1, and in a case where Z¹ represents a saturated hydrocarbon group or an unsaturated hydrocarbon group, d is an integer of 1 or more.

R⁴ represents hydrogen or a polymerizable substituent. In a case where a plurality of R⁴s is present, the plurality of R⁴s may be of an identical kind or of different kinds, and all of the plurality of R⁴s in the general formula (1) is not simultaneously hydrogen.

### BRIEF DESCRIPTION OF DRAWINGS

FIG. 1 is a cross-sectional view illustrating an example of a configuration of a hologram recording medium according to a first embodiment of the present disclosure.

FIG. 2 is a cross-sectional view illustrating an example of a configuration of a hologram recording medium according to a modification.

FIG. 3 is a schematic diagram illustrating an example of a configuration of an optical system for hologram recording.

FIG. 4 is a schematic view of an optical system for hologram recording at the time of two-beam exposure.

FIG. 5 is a cross-sectional view illustrating an example of a configuration of a hologram recording medium according to a modification.

### MODE FOR CARRYING OUT THE INVENTION

The embodiments of the present disclosure will be described in the following order.

1 Overview of Present Disclosure

2 First Embodiment (Example of Hologram Recording Medium)

3 Second Embodiment (Example of Hologram Optical Element)

4 Third Embodiment (Examples of Optical Device and Optical Unit)

5 Fourth Embodiment (Example of Method for Forming Hologram Diffraction Grating)

6 Analysis Method

### 1 Overview of Present Disclosure

First, an outline of the present disclosure will be described.

In recent years, hologram recording media are required to have a high refractive index modulation amount (Δn), a high diffraction efficiency, and high transparency, and to achieve suppression of deterioration of the storage stability in an unexposed state.

In order for a hologram recording medium to have a high refractive index modulation amount (Δn) and a high diffraction efficiency and to obtain high transparency, the photosensitive composition desirably contains a polymerizable compound that satisfies the following points.
The difference in refractive index from another material to be separated is large.
Good separation is achieved according to the light intensity difference at the time of exposure.
Little light is absorbed in the visible region.

However, an effective polymerizable compound that sufficiently satisfies the above-described points has not been found so far. Therefore, the present inventors have conducted various studies, and as a result, have found a polymerizable compound that has a specific structure and sufficiently satisfies the above-described points.

It is conventionally considered that in a hologram recording medium, a base material covering a photosensitive layer desirably has a low oxygen permeability in order to suppress inhibition, caused by oxygen in the air, of a polymerization reaction. However, as a result of studies by the present inventors, it has been found that deterioration of the storage stability in an unexposed state can be suppressed if a base material covering a photosensitive layer has an oxygen permeability larger than a specified value, instead of a low oxygen permeability as conventionally considered.

That is, the present disclosure can provide a hologram recording medium that has a high refractive index modulation amount (Δn), a high diffraction efficiency, and high transparency and can achieve suppression of deterioration of the storage stability in an unexposed state by combining a photosensitive layer containing a polymerizable compound having a specific structure with a base material having an oxygen permeability larger than a specified value, a hologram optical element, an optical device including the hologram optical element, an optical component, and a method for forming a hologram diffraction grating.

### 2 First Embodiment

[Hologram Recording Medium]

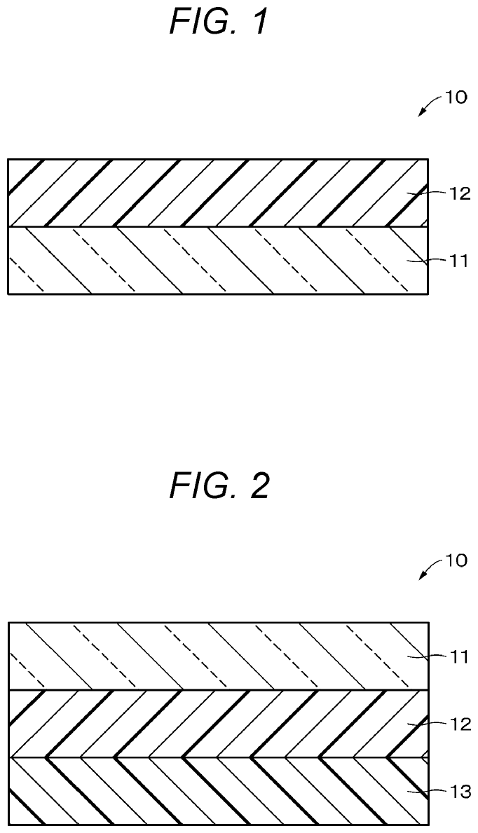

FIG. 1 is a cross-sectional view illustrating an example of a configuration of a hologram recording medium 10 according to a first embodiment of the present disclosure. The hologram recording medium 10 includes a base material 11 and a photosensitive layer 12.

(Base Material)

The base material 11 is to support the photosensitive layer 12. The base material 11 also has a function as a protective layer to protect the photosensitive layer 12. The base material 11 has transparency to visible light. The base material 11 may be a film or a substrate having rigidity, but is preferably a film from the viewpoint of increasing the oxygen permeability of the base material 11. In the present description, the film also includes a sheet. The base material 11 has a first surface on a side opposite from the photosensitive layer 12 side, and a second surface on the photosensitive layer 12 side.

The lower limit of the oxygen permeability of the base material 11 measured in an environment at 23° C. and 0% RH is more than 0.1 cm³·(m²·day·atm)⁻¹, for example, 1 cm³·(m²·day·atm)⁻¹ or more, preferably 5 cm³·(m²·day·atm)⁻¹ or more, more preferably 10 cm³·(m²·day·atm)⁻¹ or more, still more preferably 300 cm³·(m²·day·atm)⁻¹ or more, and particularly preferably 500 cm³·(m²·day·atm)⁻¹ or more. If the lower limit of the oxygen permeability of the base material 11 is 0.1 cm³·(m²·day·atm)⁻¹ or more, deterioration of the storage stability in an unexposed state can be suppressed. The upper limit of the oxygen permeability of the base material 11 measured in an environment at 23° C. and 0% RH is, for example, 10000 cm³·(m²·day·atm)⁻¹ or less, and preferably 3000 cm³·(m²·day·atm)⁻¹ or less.

The oxygen permeability of the base material 11 is measured as follows. First, the base material 11 is collected from the hologram recording medium 10. Examples of the method of collecting the base material 11 from the hologram recording medium 10 include, but are not limited to, the following methods, and any method may be used as long as the photosensitive layer 12 can be removed while damage to the base material 11 is suppressed. The base material 11 is peeled off from the photosensitive layer 12. Subsequently, the photosensitive composition remaining on the surface of the base material 11 is rubbed off using cotton, paper, or the like moistened with an organic solvent that does not affect the base material 11 or with water, and thus the base material 11 is obtained from which the photosensitive layer 12 has been removed. Alternatively, the base material 11 may be obtained from which the photosensitive layer 12 has been removed by scraping off the photosensitive composition remaining on the surface of the base material 11 using a jig such as a cutter or the like, or by peeling off the photosensitive composition remaining on the surface of the base material 11 using an adhesive tape. As the organic solvent that does not affect the base material 11, for example, an organic solvent is used such as acetone, methyl ethyl ketone, methanol, ethanol, tetrahydrofuran, toluene, methylene chloride, chloroform, or the like. Next, the oxygen permeability of the base material 11 from which the photosensitive layer 12 has been removed is measured.

Details of the measurement conditions are as follows.

Measuring device: MOCON oxygen permeability measuring device (OX-TRAN 2/21 manufactured by Hitachi High-Tech Science Corporation)

Environment set in measuring device: measurement temperature set at 23° C., measurement humidity set at 0% RH

Measurement method: JIS K 7126-1 or JIS K 7126-2

The base material 11 contains, for example, a polymer material. The polymer material contains, for example, at least one selected from the group consisting of cycloolefin-based resins, polycarbonate-based resins, polyester-based resins, and cellulose-based resins. In a case where the base material 11 contains two or more resins, the two or more resins may be mixed, copolymerized, or stacked to form a laminated film.

Examples of the cycloolefin-based resins include norbornene-based polymers, vinyl alicyclic hydrocarbon polymers, cyclic conjugated diene polymers, and the like. Among them, norbornene-based polymers are preferable. Examples of the norbornene-based polymers include ring-opened polymers of norbornene-based monomers, norbornene-based copolymers obtained by copolymerizing a norbornene-based monomer and an α-olefin such as ethylene or the like, and the like. Examples of the polycarbonate-based resins include aliphatic polycarbonate-based resins, aromatic polycarbonate-based resins, and the like. Examples of the polyester-based resins include polyethylene terephthalate-based resins. Examples of the cellulose-based resins include acetyl cellulose-based resins such as triacetyl cellulose (TAC), diacetyl cellulose, and the like.

The thickness of the base material 11 may be appropriately set by those skilled in the art, but is preferably 0.1 μm or more and 200 μm or less, more preferably 1 μm or more and 150 μm or less, and still more preferably 3 μm or more and 70 μm or less from the viewpoint of transparency and rigidity of the hologram recording medium 10.

The base material 11 may include a hard coat layer as necessary. The hard coat layer may be provided on both of the first surface and the second surface of the base material 11 or on one surface of the base material 11. In a case where the base material 11 includes a hard coat layer, the oxygen permeability of the base material 11 is measured in a state where the hard coat layer is included. The hard coat layer contains, for example, an ultraviolet curable resin. The hard coat layer may contain an additive such as fine particles or the like as necessary.

(Photosensitive Layer)

The photosensitive layer 12 contains a photosensitive composition. The photosensitive composition contains a polymerizable compound and a photopolymerization initiator. The photosensitive composition may further contain a binder resin.

The thickness of the photosensitive layer 12 may be appropriately set by those skilled in the art, but is preferably 0.1 μm or more and 100 μm or less, and more preferably 1 μm or more and 30 μm or less from the viewpoint of diffraction efficiency and sensitivity to light. The photosensitive layer 12 has a first surface on the base material 11 side, and a second surface on a side opposite from the base material 11 side.

Hereinafter, each component in the photosensitive composition will be described in detail.

(Polymerizable Compound)

The polymerizable compound contains a compound represented by the general formula (1) described below. The compound has a high refractive index, good transparency, and good solubility in an organic solvent.

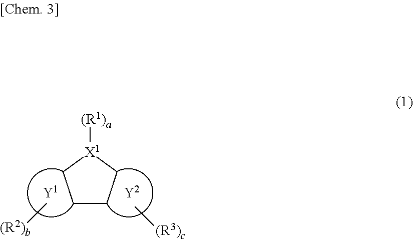

In the general formula (1), X¹ represents an oxygen atom, a nitrogen atom, a phosphorus atom, a carbon atom, or a silicon atom. In a case where X¹ represents an oxygen atom, a is 0, in a case where X¹ represents a nitrogen atom or a phosphorus atom, a is 1, and in a case where X¹ represents a carbon atom or a silicon atom, a is 2.

Y¹ and Y² each represent a benzene ring or a naphthalene ring. Y¹ and Y² may simultaneously represent a benzene ring, or a case may be excluded in which Y¹ and Y² simultaneously represent a benzene ring. In a case where Y¹ and/or Y² represents a benzene ring, b or c corresponding to Y¹ and/or Y² representing the benzene ring is 4. In a case where Y¹ and/or Y² represents a naphthalene ring, b and/or c corresponding to Y¹ and/or Y² representing the naphthalene ring is 6.

R¹ to R³ each represent hydrogen or a substituent represented by *—Z¹(R⁴)_{d} (in which * represents a binding site). In a case where a plurality of R¹s, a plurality of R²s, and a plurality of R³s are present, the plurality of R¹s, the plurality of R²s, and the plurality of R³s may be of an identical kind or of different kinds, and all of the plurality of R¹s, the plurality of R²s, and the plurality of R³s in the general formula (1) are not simultaneously hydrogen.

Z¹ represents a single bond, a divalent or higher saturated hydrocarbon group, or a divalent or higher unsaturated hydrocarbon group, and the saturated hydrocarbon group or the unsaturated hydrocarbon group may contain an ether bond and/or a thioether bond. In a case where Z¹ represents a single bond, d is 1, and in a case where Z¹ represents a saturated hydrocarbon group or an unsaturated hydrocarbon group, d is an integer of 1 or more.

R⁴ represents hydrogen or a polymerizable substituent. In a case where a plurality of R⁴s is present, the plurality of R⁴s may be of an identical kind or of different kinds, and all of the plurality of R⁴s in the general formula (1) is not simultaneously hydrogen.

In the general formula (1), X¹ represents an oxygen atom, a nitrogen atom, a phosphorus atom, a carbon atom, or a silicon atom. Furthermore, it is considered that an effect of the present disclosure can also be expected for Group 14 elements, Group 15 elements, and Group 16 elements (however, transition metals are excluded) other than the above-described atoms.

Among the above-described atoms, an oxygen atom, a nitrogen atom, and a carbon atom, which are representative elements in an organic compound, are preferable from the viewpoint of ease of synthesis of a compound, and the atomic refraction of each atom is as follows. Oxygen atom: 1.6 to 2.2, nitrogen atom: 3.5 to 4.4, carbon atom: 1.7 to 2.4 (Optical Review, Vol. 44, No. 8, 2015, p. 298-303). In the present embodiment, X¹ in the general formula (1) is preferably a nitrogen atom, which has a high value of atomic refraction, from the viewpoint of obtaining a compound having a high refractive index.

That is, in the present embodiment, the polymerizable compound can have the following structures.

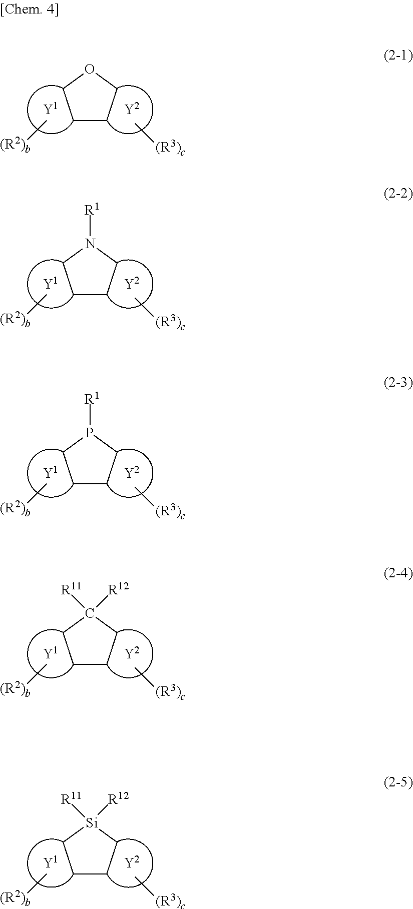

In the general formulas (2-1) to (2-5), Y¹ and Y² each represent a benzene ring or a naphthalene ring. Y¹ and Y² may simultaneously represent a benzene ring, or a case may be excluded in which Y¹ and Y² simultaneously represent a benzene ring. In a case where Y¹ and/or Y² represents a benzene ring, b or c corresponding to Y¹ and/or Y² representing the benzene ring is 4. In a case where Y¹ and/or Y² represents a naphthalene ring, b and/or c corresponding to Y¹ and/or Y² representing the naphthalene ring is 6.

R¹, R², R³, R¹¹, and R¹² each represent hydrogen or a substituent represented by *—Z¹(R⁴)_{d} (in which * represents a binding site). In a case where a plurality of R¹s, a plurality of R²s, and a plurality of R³s are present, the plurality of R¹s, the plurality of R²s, and the plurality of R³s may be of an identical kind or of different kinds, and all of R¹s, R²s, R³s, R¹¹s, and R¹²s in the general formulas (2-1) to (2-5) are not simultaneously hydrogen.

Z¹ represents a single bond, a divalent or higher saturated hydrocarbon group, or a divalent or higher unsaturated hydrocarbon group, and the saturated hydrocarbon group or the unsaturated hydrocarbon group may contain an ether bond and/or a thioether bond. In a case where Z¹ represents a single bond, d is 1, and in a case where Z¹ represents a saturated hydrocarbon group or an unsaturated hydrocarbon group, d is an integer of 1 or more.

R⁴ represents hydrogen or a polymerizable substituent. In a case where a plurality of R⁴s is present, the plurality of R⁴s may be of an identical kind or of different kinds, and all of the plurality of R⁴s in the general formulas (2-1) to (2-5) is not simultaneously hydrogen.

Furthermore, in the general formula (1), Y¹ and Y² each represent a benzene ring or a naphthalene ring. Y¹ and Y² may simultaneously represent a benzene ring, or a case may be excluded in which Y¹ and Y² simultaneously represent a benzene ring.

The molecular refraction of phenyl (C₆H₅) and naphthyl (C₁₀H₇) is as follows. Phenyl (C₆H₅): 25.5, naphthyl (C₁₀H₇): 43.3 (Optical Review, Vol. 44, No. 8, 2015, p. 298-303). In the present embodiment, Y¹ and Y² each more preferably represent a naphthalene ring, which has a high value of molecular refraction, from the viewpoint of obtaining a compound having a high refractive index.

That is, in the present embodiment, the polymerizable compound can have the following structures.

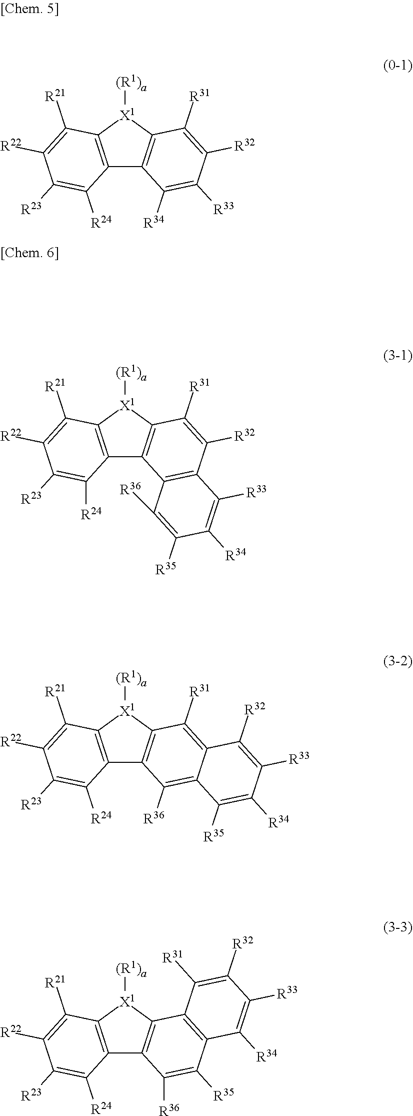

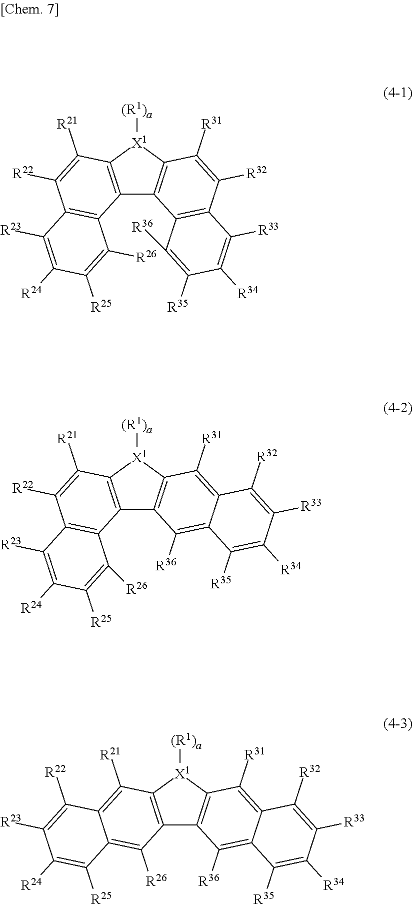

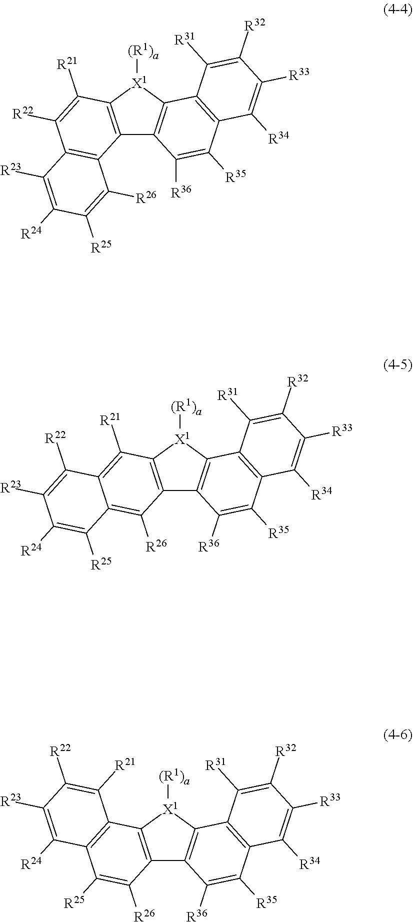

In the general formulas (0-1), (3-1) to (3-3), and (4-1) to (4-6), X¹ represents an oxygen atom, a nitrogen atom, a phosphorus atom, a carbon atom, or a silicon atom. In a case where X¹ represents an oxygen atom, a is 0, in a case where X¹ represents a nitrogen atom or a phosphorus atom, a is 1, and in a case where X¹ represents a carbon atom or a silicon atom, a is 2.

R¹, R²¹ to R²⁶, and R³¹ to R³⁶ each represent hydrogen or a substituent represented by *—Z¹(R⁴)_{d} (in which * represents a binding site). R¹, R²¹ to R²⁶, and R³¹ to R³⁶ may be of an identical kind or of different kinds. Furthermore, in a case where a plurality of R¹s is present, the plurality of R¹s may of an identical kind or of different kinds. However, R¹, R²¹ to R²⁶, and R³¹ to R³⁶ in the general formulas (0-1), (3-1) to (3-3), and (4-1) to (4-6) are not simultaneously hydrogen.

Z¹ represents a single bond, a divalent or higher saturated hydrocarbon group, or a divalent or higher unsaturated hydrocarbon group, and the saturated hydrocarbon group or the unsaturated hydrocarbon group may contain an ether bond and/or a thioether bond. In a case where Z¹ represents a single bond, d is 1, and in a case where Z¹ represents a saturated hydrocarbon group or an unsaturated hydrocarbon group, d is an integer of 1 or more.

R⁴ represents hydrogen or a polymerizable substituent. In a case where a plurality of R⁴s is present, the plurality of R⁴s may be of an identical kind or of different kinds, and all of the plurality of R⁴s in the general formulas (0-1), (3-1) to (3-3), and (4-1) to (4-6) is not simultaneously hydrogen.

In the general formula (1), Z¹ represents a single bond, a divalent or higher saturated hydrocarbon group, or a divalent or higher unsaturated hydrocarbon group. The saturated hydrocarbon group or the unsaturated hydrocarbon group may contain an ether bond and/or a thioether bond.

In a case where Z¹ is a divalent or higher saturated hydrocarbon group, the saturated hydrocarbon group may be a linear, branched, or cyclic substituted or unsubstituted hydrocarbon group. In general, the larger simple carbon chain number an organic compound has, the more easily the organic compound tends to obtain solubility. Meanwhile, the larger simple carbon chain number an organic compound has, the lower refractive index the organic compound tends to have. Therefore, the saturated hydrocarbon group preferably has a simple carbon chain number of 1 to 15, and more preferably 1 to 10.

Furthermore, in a case where Z¹ is a divalent or higher unsaturated hydrocarbon group, the unsaturated hydrocarbon group may be a linear, branched, or cyclic substituted or unsubstituted hydrocarbon group or an aromatic group. The unsaturated hydrocarbon group preferably has a simple carbon chain number of 1 to 15, and more preferably 1 to 10. In a case where the unsaturated hydrocarbon group contains an aromatic group, the aromatic group is preferably a substituted or unsubstituted divalent or higher aromatic group represented by the chemical formulas (5-1) to (5-8) described below. Four or more benzene rings linearly connected absorb light in the visible light region and have a color, and therefore may be undesirable from the viewpoint of transparency. Therefore, the aromatic group preferably does not have a structure in which four or more benzene rings are linearly arranged, and the aromatic group preferably has a linear shape including a benzene ring, a naphthalene ring, or an anthracene ring.

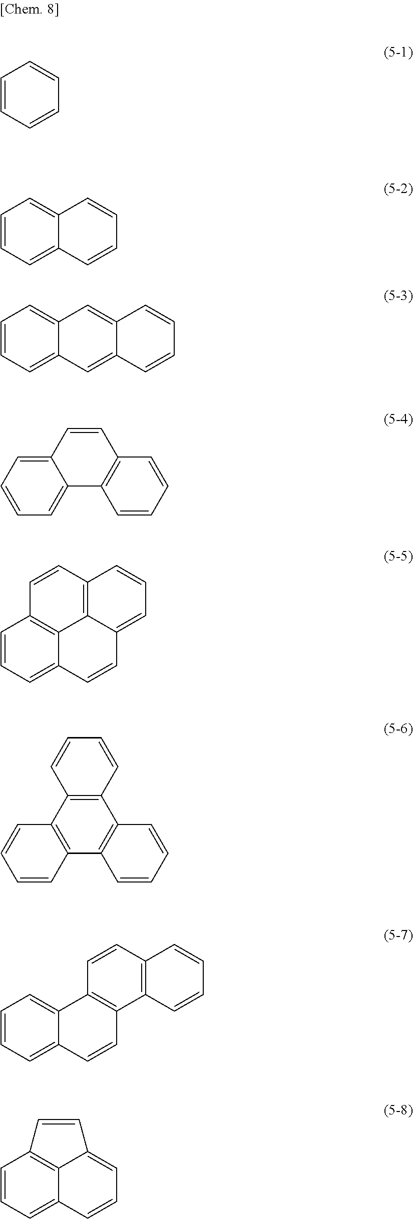

In the general formula (1), examples of the polymerizable substituent represented by R⁴ include those including a polymerizable unsaturated group and those including a reactive substituent. Examples of the polymerizable substituents including a polymerizable unsaturated group include a vinyl group, an acrylic group, a methacrylic group, an acrylamide group, a methacrylamide group, a cyanoacrylate group, a cyanomethacrylate group, a vinyl ether group, a vinyl cyanide group, a vinyl nitrate group, conjugated polyene groups, vinyl halide groups, vinyl ketone groups, a styryl group, and the like. Examples of the polymerizable substituents including a reactive substituent include an epoxy group, an oxetane group, a hydroxyl group, an amino group, a carboxyl group, acid anhydride groups, acid halide groups, an isocyanate group, and the like.

In the general formula (1), X¹ preferably represents a nitrogen atom, and Y¹ and Y² each preferably represent a naphthalene ring. That is, the above-described compound is preferably represented by the general formula (1-1) described below.

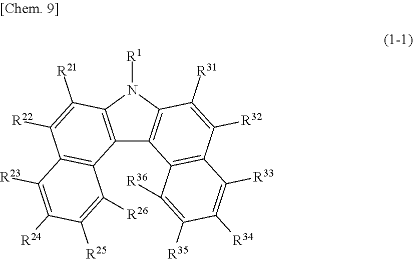

In the general formula (1-1), R¹, R²¹ to R²⁶, and R³¹ to R³⁶ represent hydrogen or a substituent represented by *—Z¹(R⁴)_{d} (in which * represents a binding site). R¹, R²¹ to R²⁶, and R³¹ to R³⁶ may be of an identical kind or of different kinds. However, R¹, R²¹ to R²⁶, and R³¹ to R³⁶ are not simultaneously hydrogen.

Z¹ represents a single bond, a divalent or higher saturated hydrocarbon group, or a divalent or higher unsaturated hydrocarbon group, and the saturated hydrocarbon group or the unsaturated hydrocarbon group may contain an ether bond and/or a thioether bond. In a case where Z¹ represents a single bond, d is 1, and in a case where Z¹ represents a saturated hydrocarbon group or an unsaturated hydrocarbon group, d is an integer of 1 or more.

R⁴ represents hydrogen or a polymerizable substituent. In a case where a plurality of R⁴s is present, the plurality of R⁴s may be of an identical kind or of different kinds, and all of the plurality of R⁴s in the general formula (1-1) is not simultaneously hydrogen.

In the general formula (1-1), R¹ is preferably a substituent represented by *—Z¹(R⁴)_{d} (in which * represents a binding site), and R²¹ to R²⁶ and R³¹ to R³⁶ preferably represent hydrogen.

Furthermore, in the general formula (1), X¹ preferably represents a carbon atom, and Y¹ and Y² each preferably represent a naphthalene ring. That is, the above-described compound is preferably represented by the general formula (1-2) described below.

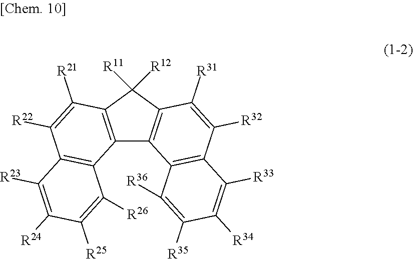

In the general formula (1-2), R¹¹, R¹², R²¹ to R²⁶, and R³¹ to R³⁶ represent hydrogen or a substituent represented by *—Z¹(R⁴)_{d} (in which * represents a binding site). R¹¹, R¹², R²¹ to R²⁶, and R³¹ to R³⁶ may be of an identical kind or of different kinds. However, R¹¹, R¹², R²¹ to R²⁶, and R³¹ to R³⁶ in the general formula (1-2) are not simultaneously hydrogen.

Z¹ represents a single bond, a divalent or higher saturated hydrocarbon group, or a divalent or higher unsaturated hydrocarbon group, and the saturated hydrocarbon group or the unsaturated hydrocarbon group may contain an ether bond and/or a thioether bond. In a case where Z¹ represents a single bond, d is 1, and in a case where Z¹ represents a saturated hydrocarbon group or an unsaturated hydrocarbon group, d is an integer of 1 or more.

R⁴ represents hydrogen or a polymerizable substituent. In a case where a plurality of R⁴s is present, the plurality of R⁴s may be of an identical kind or of different kinds, and all of the plurality of R⁴s in the general formula (1-2) is not simultaneously hydrogen.

In the general formula (1-2), R¹¹ and/or R¹² is preferably a substituent represented by *—Z¹(R⁴)_{d} (in which * represents a binding site), and R²¹ to R²⁶ and R³¹ to R³⁶ preferably represent hydrogen.

A preferred monofunctional exemplary compound of the polymerizable compound of the present embodiment has the following chemical formula.

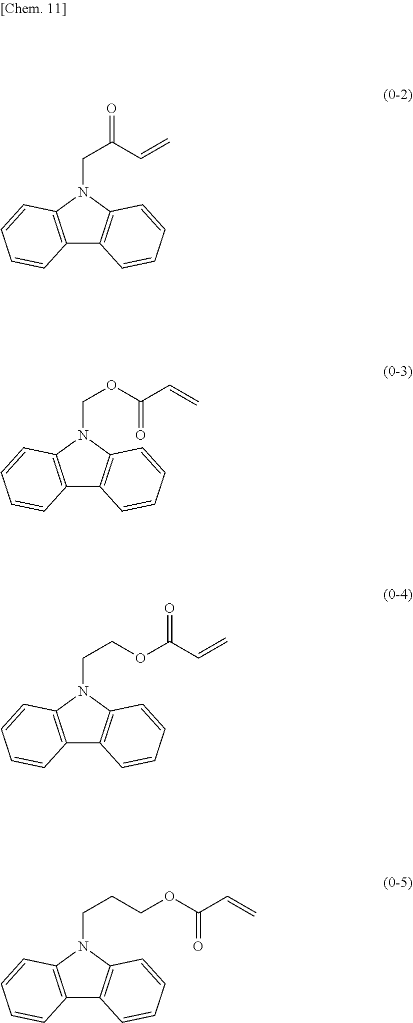 [SMILES: C=CC(=O)Cn1c2ccccc2c2ccccc21 ; C=CC(=O)OCn1c2ccccc2c2ccccc21 ; C=CC(=O)OCCn1c2ccccc2c2ccccc21 ; C=CC(=O)OCCCn1c2ccccc2c2ccccc21]

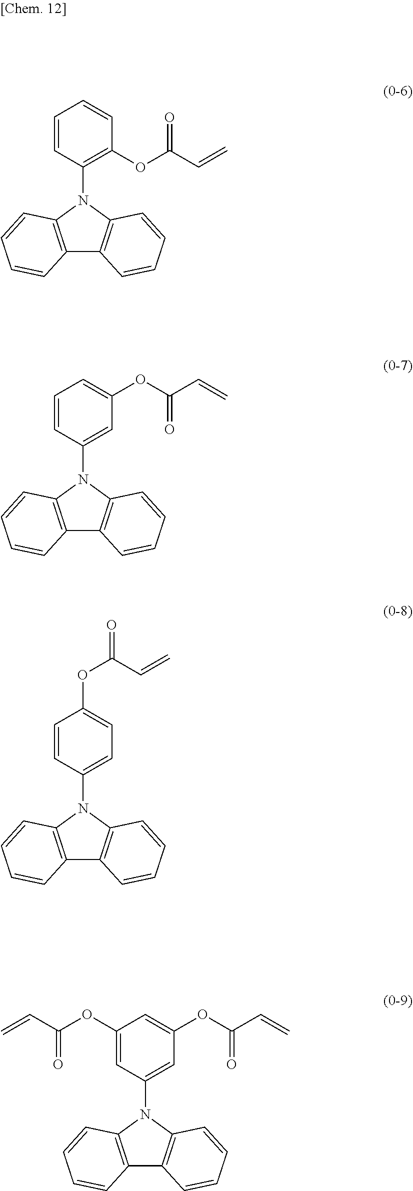

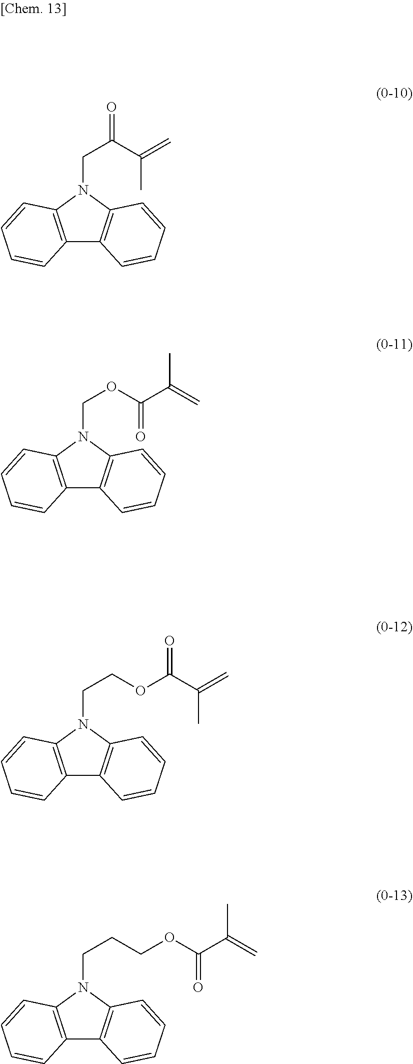 [SMILES: C=C(C)C(=O)Cn1c2ccccc2c2ccccc21 ; C=C(C)C(=O)OCn1c2ccccc2c2ccccc21 ; C=C(C)C(=O)OCCn1c2ccccc2c2ccccc21 ; C=C(C)C(=O)OCCCn1c2ccccc2c2ccccc21]

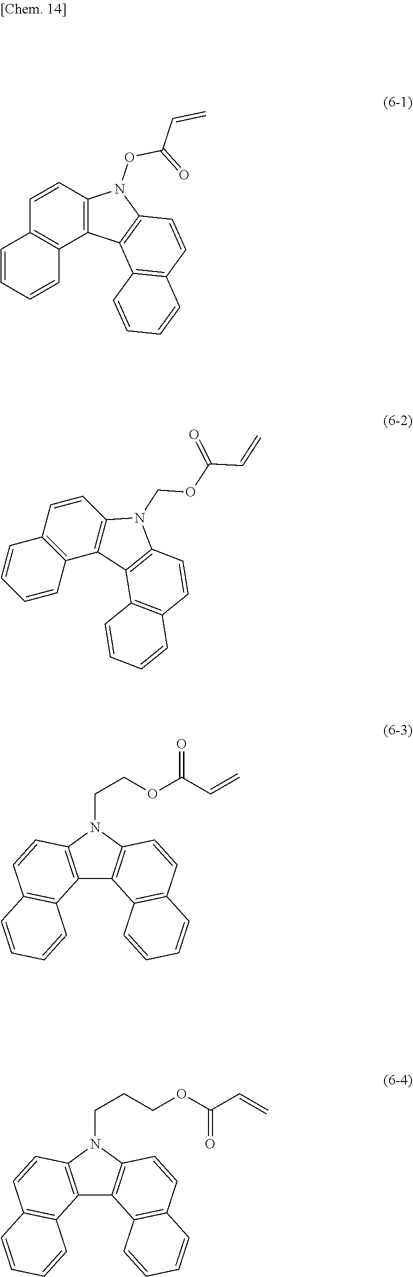 [SMILES: C=CC(=O)On1c2ccc3ccccc3c2c2c3ccccc3ccc21 ; C=CC(=O)OCn1c2ccc3ccccc3c2c2c3ccccc3ccc21 ; C=CC(=O)OCCn1c2ccc3ccccc3c2c2c3ccccc3ccc21 ; C=CC(=O)OCCCn1c2ccc3ccccc3c2c2c3ccccc3ccc21]

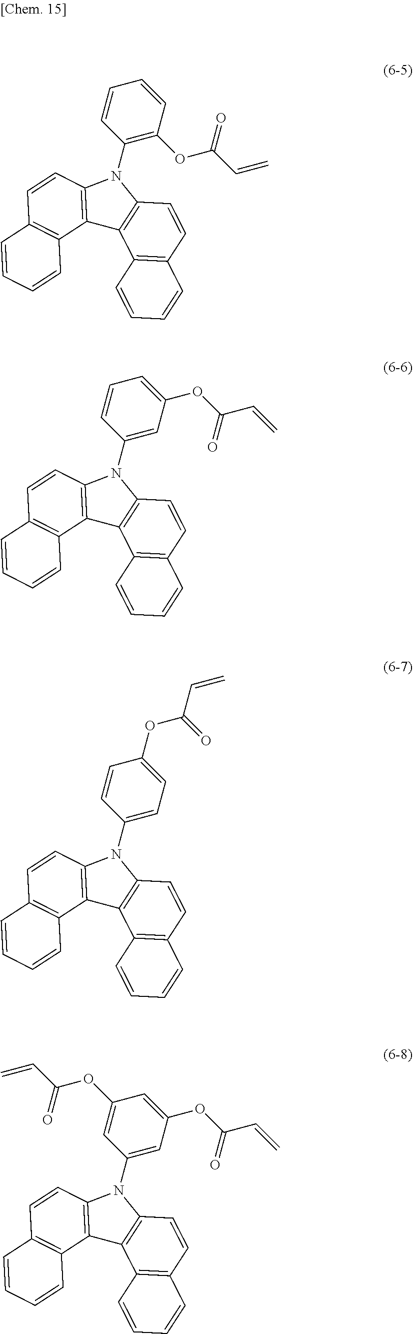

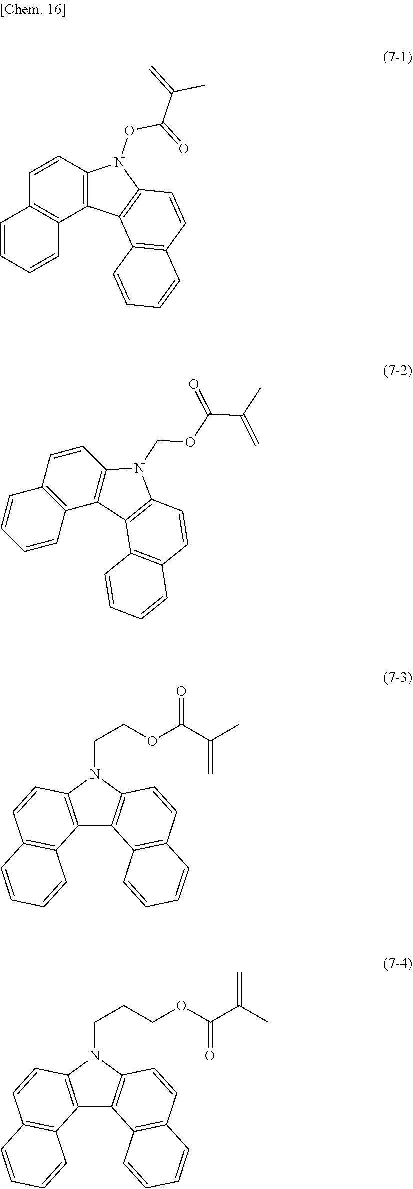 [SMILES: C=C(C)C(=O)On1c2ccc3ccccc3c2c2c3ccccc3ccc21 ; C=C(C)C(=O)OCn1c2ccc3ccccc3c2c2c3ccccc3ccc21 ; C=C(C)C(=O)OCCn1c2ccc3ccccc3c2c2c3ccccc3ccc21 ; C=C(C)C(=O)OCCCn1c2ccc3ccccc3c2c2c3ccccc3ccc21]

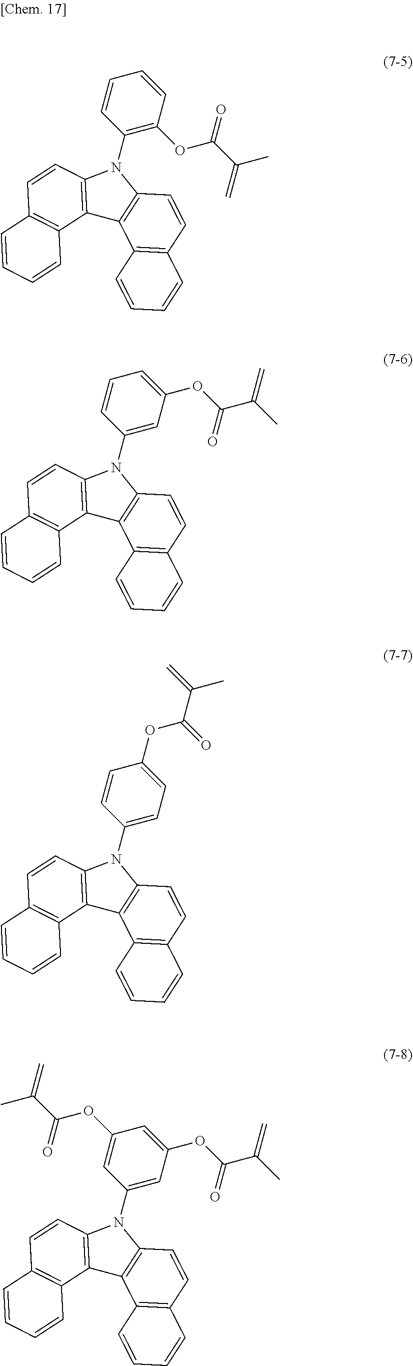

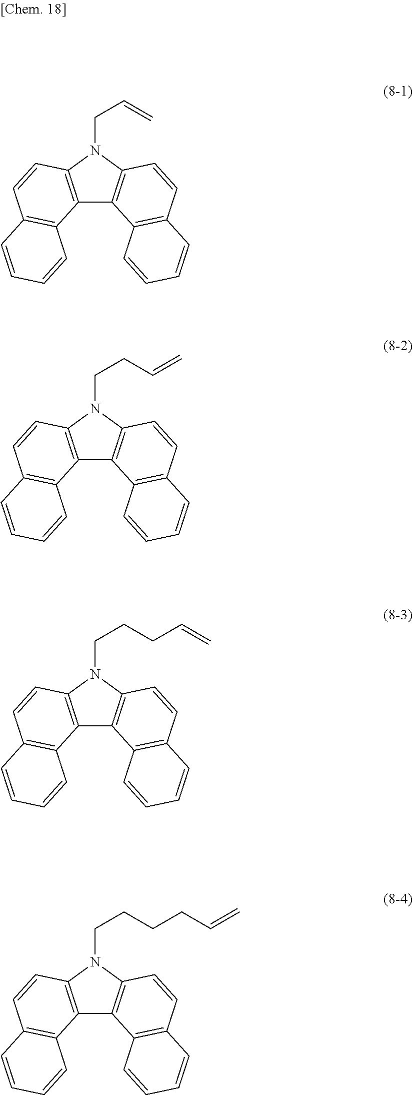 [SMILES: C=CCn1c2ccc3ccccc3c2c2c3ccccc3ccc21 ; C=CCCn1c2ccc3ccccc3c2c2c3ccccc3ccc21 ; C=CCCCn1c2ccc3ccccc3c2c2c3ccccc3ccc21 ; C=CCCCCn1c2ccc3ccccc3c2c2c3ccccc3ccc21]

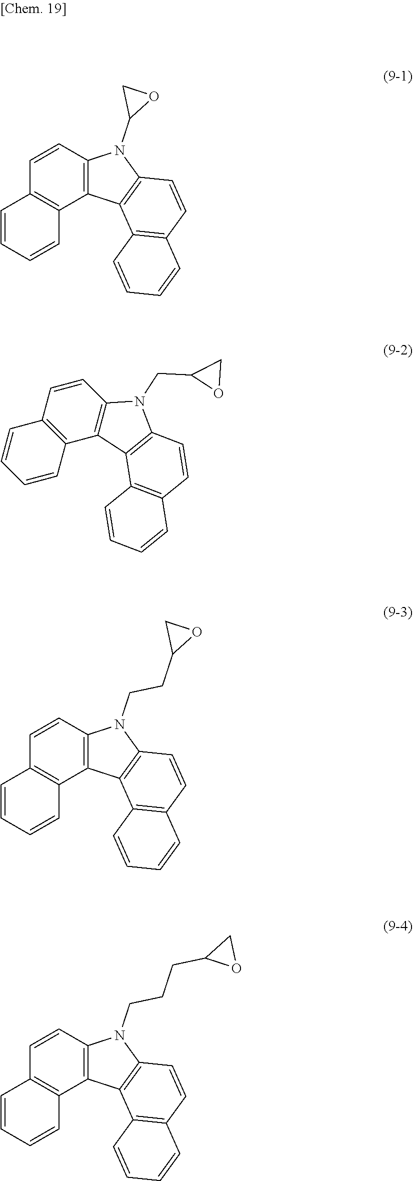 [SMILES: c1ccc2c(c1)ccc1c2c2c3ccccc3ccc2n1C1CO1 ; c1ccc2c(c1)ccc1c2c2c3ccccc3ccc2n1CC1CO1 ; c1ccc2c(c1)ccc1c2c2c3ccccc3ccc2n1CCC1CO1 ; c1ccc2c(c1)ccc1c2c2c3ccccc3ccc2n1CCCC1CO1]

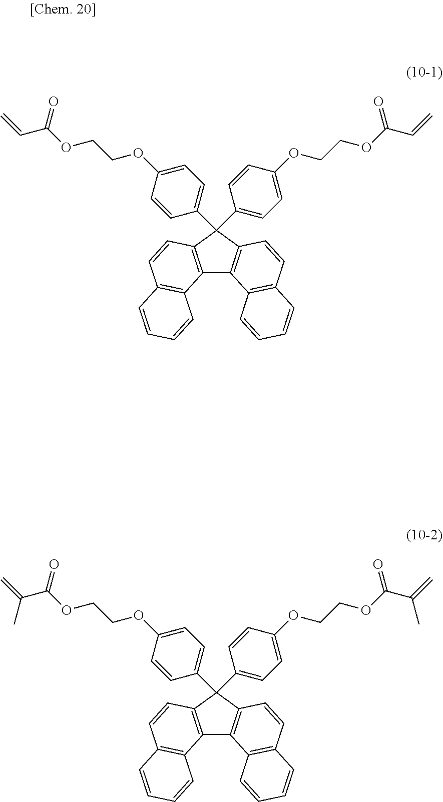

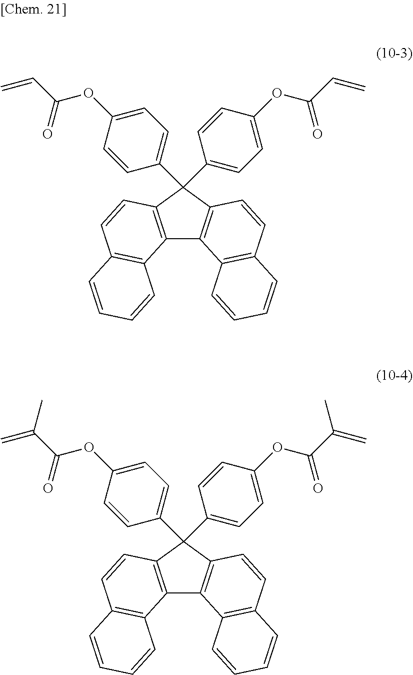

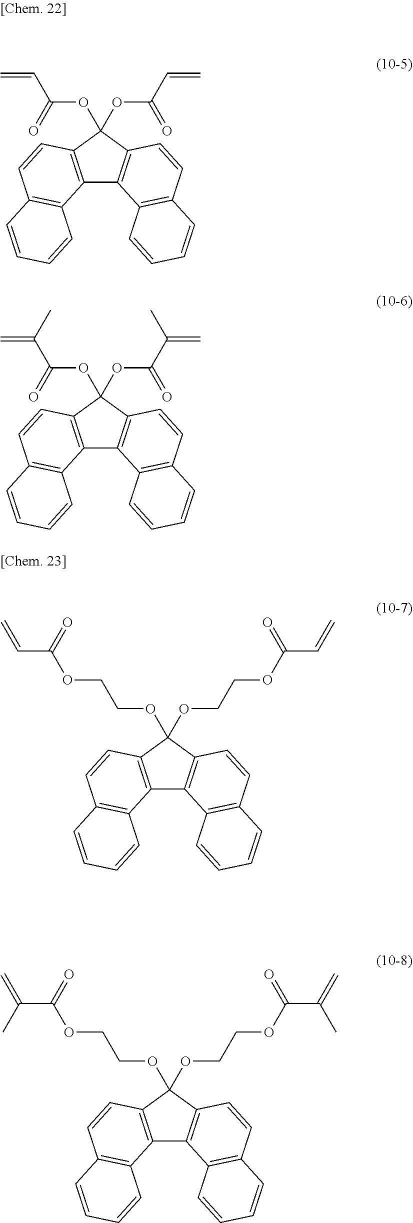

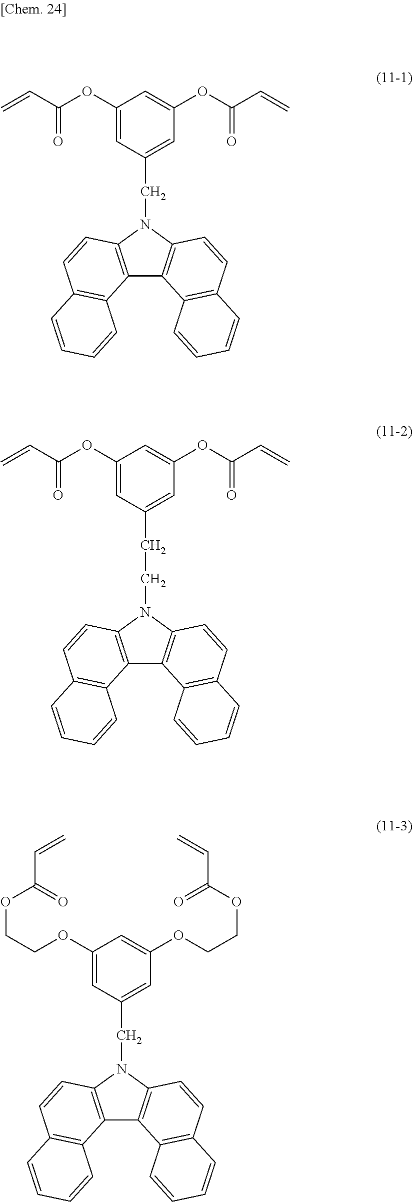

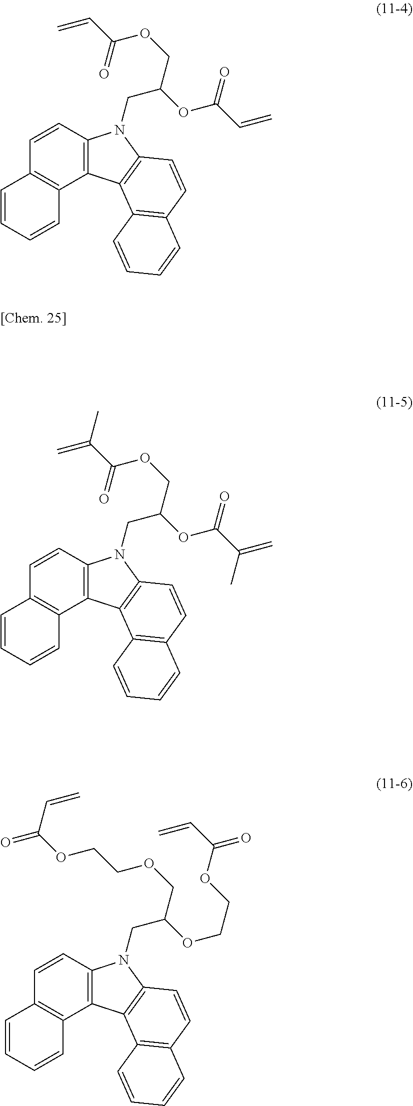 [SMILES: C=CC(=O)OCC(Cn1c2ccc3ccccc3c2c2c3ccccc3ccc21)OC(=O)C=C ; C=C(C)C(=O)OCC(Cn1c2ccc3ccccc3c2c2c3ccccc3ccc21)OC(=O)C(=C)C ; C=CC(=O)OCCOCC(Cn1c2ccc3ccccc3c2c2c3ccccc3ccc21)OCCOC(=O)C=C]

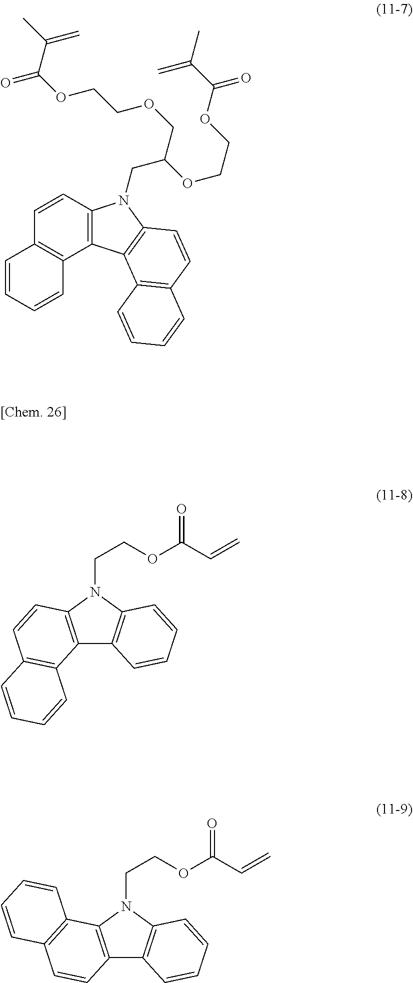 [SMILES: C=C(C)C(=O)OCCOCC(Cn1c2ccc3ccccc3c2c2c3ccccc3ccc21)OCCOC(=O)C(=C)C ; C=CC(=O)OCCn1c2ccccc2c2c3ccccc3ccc21 ; C=CC(=O)OCCn1c2ccccc2c2ccc3ccccc3c21]

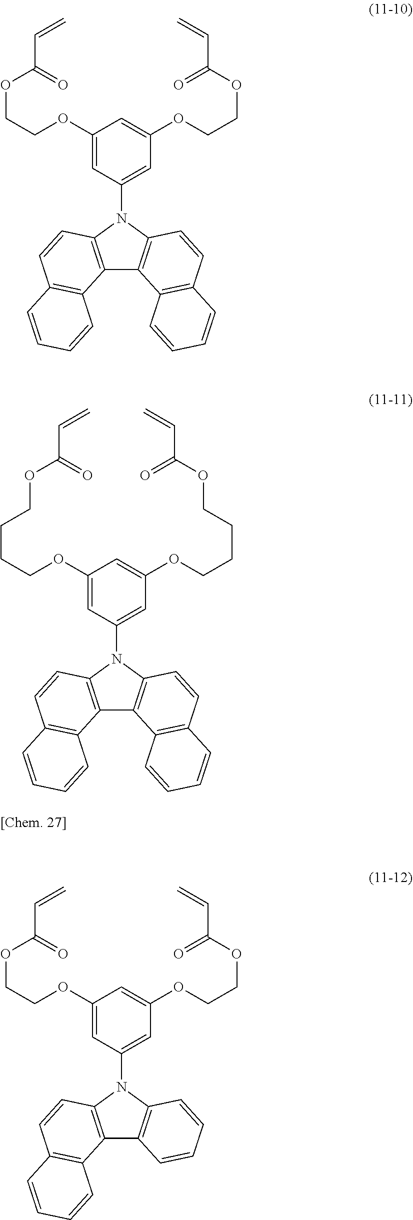 [SMILES: C=CC(=O)OCCOc1cc(OCCOC(=O)C=C)cc(-n2c3ccc4ccccc4c3c3c4ccccc4ccc32)c1 ; C=CC(=O)OCCCCOc1cc(OCCCCOC(=O)C=C)cc(-n2c3ccc4ccccc4c3c3c4ccccc4ccc32)c1 ; C=CC(=O)OCCOc1cc(OCCOC(=O)C=C)cc(-n2c3ccccc3c3c4ccccc4ccc32)c1]

In the present embodiment, the polymerizable compound preferably has a refractive index of 1.60 or more, more preferably 1.65 or more, and still more preferably 1.70 or more. Meanwhile, the refractive index of the polymerizable compound is, for example, 1.80 or less, but may be more than 1.80.

Note that the refractive index can be measured with a critical angle method or a spectroscopic ellipsometry method. For example, in the critical angle method, the refractive index can be measured using an Abbe refractometer ER-1 manufactured by ERMA INC. (using a measurement wavelength of 486 nm, 589 nm, 656 nm, or the like in the visible light region).

(Photopolymerization Initiator)

The photopolymerization initiator is not particularly limited, and any photopolymerization initiator can be used. Examples of the photopolymerization initiator may include radical polymerization initiators (radical generators), cationic polymerization initiators (acid generators), and polymerization initiators having both of the functions of a radical generator and an acid generator. Note that an anionic polymerization initiator (base generator) may be used as the photopolymerization initiator.

The radical polymerization initiator (radical generator) preferably contains at least one of an organic boron salt-based initiator or an onium salt-based initiator, and more preferably contains both of an organic boron salt-based initiator and an onium salt-based initiator from the viewpoint of obtaining a high refractive index modulation amount (Δn) and a high diffraction efficiency. Examples of the organic boron salt-based initiator being available include tetrabutylammonium butyltriphenylborate (manufactured by Showa Denko K.K., product name: P3B) and tetrabutylammonium=butyltrinaphthylborate (manufactured by Showa Denko K.K., product name: N3B). Examples of the onium salt-based initiator being available include 4-isopropyl-4′-methyldiphenyliodonium tetrakis(pentafluorophenyl)borate (manufactured by Tokyo Chemical Industry Co., Ltd., product code: I0591), (2-methylphenyl) (2,4,6-trimethylphenyl)iodonium trifluoromethanesulfonate mesityl(o-tolyl)iodonium triflate (manufactured by Tokyo Chemical Industry Co., Ltd., product code: M2907), and (4-methylphenyl) (2,4,6-trimethylphenyl)iodonium trifluoromethanesulfonate mesityl(p-tolyl)iodonium triflate (manufactured by Tokyo Chemical Industry Co., Ltd., product code: M2909), which are iodonium salts, and tri-p-tolylsulfonium hexafluorophosphate (manufactured by Tokyo Chemical Industry Co., Ltd., product code: T2041), diphenyl 4-thiophenoxyphenylsulfonium hexafluorophosphate (manufactured by San-Apro Ltd., product name: CPI-100P), a triarylsulfonium borate salt (manufactured by San-Apro Ltd., product name: CPI-310B), diphenyl 4-thiophenoxyphenylsulfonium tetrakis(pentafluorophenyl)borate (manufactured by San-Apro Ltd., product name: CPI-100B), a diphenyl 4-thiophenoxyphenylsulfonium fluorinated alkylfluorophosphate salt (manufactured by San-Apro Ltd., product name: CPI-200K), and a triarylsulfonium borate salt (manufactured by BASF Japan, product name: Irgacure 290), which are sulfonium salts.

(Binder Resin)

The binder resin can be effective for improving the film strength and improving the heat resistance and the mechanical strength. The binder resin is not particularly limited, and any binder resin can be used.

Examples of the binder resin include vinyl acetate-based resins such as polyvinyl acetate, its hydrolyzates, and the like; acrylic resins such as poly(meth)acrylic acid esters, partial hydrolyzates thereof, and the like; polyvinyl alcohol and its partial acetalized products; triacetyl cellulose; polyisoprene; polybutadiene; polychloroprene; silicone rubber; polystyrene; polyvinyl butyral; polychloroprene; polyvinyl chloride; polyarylate; chlorinated polyethylene; chlorinated polypropylene; poly-N-vinylcarbazole and its derivatives; poly-N-vinylpyrrolidone and its derivatives; polyarylate; copolymers of styrene and maleic anhydride and half-esters thereof; copolymers containing at least one in the copolymerizable monomer group including acrylic acid, acrylic acid esters, methacrylic acid, methacrylic acid esters, acrylamide, acrylonitrile, ethylene, propylene, vinyl chloride, vinyl acetate, and the like as a polymerization component; and the like, and one or more of these can be used. Moreover, as the copolymerization component, a monomer can also be used that contains a curable functional group that can be cured by heat or light.

Furthermore, an oligomer-type curable resin can also be used as the binder resin. Examples of the oligomer-type curable resin include epoxy compounds produced by condensation reactions of various phenol compounds such as bisphenol A, bisphenol S, novolac, o-cresol novolac, p-alkylphenol novolac, and the like with epichlorohydrin, and one or more of these can be used.

(Other Components)

The photosensitive composition may contain, in addition to the above-described components, at least one selected from the group consisting of radical polymerizable monomers other than the compounds represented by the general formula (1) described above, sensitizing dyes, inorganic fine particles, plasticizers, chain transfer agents, polymerization inhibitors, UV sensitizers, and the like.

Examples of the radical polymerizable monomers other than the compounds represented by the general formula (1) include monofunctional or polyfunctional carbazole-based monomers, dinaphthothiophene-based monomers, fluorene-based monomers, dibenzofuran-based monomers, and the like, and one or more of these can be used.

The sensitizing dye can increase the sensitivity of the photopolymerization initiator to light. The sensitizing dye may contain either or both of a dye that absorbs light in the visible light region and a UV sensitizing dye to be added for the purpose of improving light efficiency at the time of UV irradiation (such as an anthracene compound or the like). Furthermore, only one kind of sensitizing dye may be used, or a plurality of kinds of sensitizing dyes may be used to respond to a plurality of wavelengths.

The sensitizing dye is not particularly limited, and examples of the sensitizing dye include thiopyrylium salt-based dyes, merocyanine-based dyes, quinoline-based dyes, rose bengal-based dyes, styryl quinoline-based dyes, ketocoumarin-based dyes, thioxanthene-based dyes, xanthene-based dyes, thiazine-based dyes, azine-based dyes, phenazine-based dyes, oxonol-based dyes, cyanine-based dyes, rhodamine-based dyes, pyrylium salt-based dyes, cyclopentanone-based dyes, cyclohexanone-based dyes, and the like. Specific examples of the cyanine and merocyanine-based dyes include 3,3′-dicarboxyethyl-2,2′-thiocyanine bromide, 1-carboxymethyl-1′-carboxyethyl-2,2′-quinocyanine bromide, 1,3′-diethyl-2,2′-quinothiacyanine iodide, 3-ethyl-5-[(3-ethyl-2(3H)-benzothiazolylidene)ethylidene]-2-thioxo-4-oxazolidine, and the like, specific examples of the coumarin and ketocoumarin-based dyes include 3-(2′-benzimidazole)-7-diethylaminocoumarin, 3,3′-carbonylbis(7-diethylaminocoumarin), 3,3′-carbonylbiscoumarin, 3,3′-carbonylbis(5,7-dimethoxycoumarin), 3,3′-carbonylbis(7-acetoxycoumarin), 2,3-dihydro-1,3,3-trimethyl-2-[2-(2-methyl-3H-indole-3-ylidene)ethylidene]-1H-indole, and the like, specific examples of the thiazine-based dyes include methylene blue and the like, specific examples of the azine-based dyes include Safranin 0 and the like, and specific examples of the cyclopentanone-based dyes include (2E,5E)-2,5-bis[(4-(dimethylamino)phenyl)methylene]cyclopentanone, (2E,5E)-2,5-bis[(4-(diethylamino)phenyl)methylene]cyclopentanone, and the like, and one or more of these can be used.

As the inorganic fine particles, for example, TiO₂ fine particles or ZrO₂ fine particles can be used. The photosensitive composition in the present embodiment may contain one kind of inorganic fine particles or two or more kinds of inorganic fine particles. For example, the TiO₂ fine particles and the ZrO₂ fine particles may be used in combination.

The plasticizer is effective for adjustment of the adhesion, the flexibility, the hardness, and other physical characteristics of the photosensitive composition.

The plasticizer is not particularly limited, and examples of the plasticizer include triethylene glycol, triethylene glycol diacetate, triethylene glycol dipropionate, triethylene glycol dicaprylate, triethylene glycol dimethyl ether, poly(ethylene glycol), poly(ethylene glycol) methyl ether, triethylene glycol bis(2-ethylhexanoate), tetraethylene glycol diheptanoate, diethyl sebacate, dibutyl suberate, tris(2-ethylhexyl)phosphate, isozorobyl naphthalene, diisopropyl naphthalene, poly(propylene glycol), glyceryl tributyrate, diethyl adipate, diethyl sebacate, monobutyl suberate, tributyl phosphate, tris(2-ethylhexyl)phosphate, and the like, and one or more of these can be used.

Furthermore, the plasticizer may have a polymerizable reactive group. The plasticizer is not particularly limited, and for example, a cationic polymerizable compound can be used. The cationic polymerizable compound is not particularly limited, and examples of the cationic polymerizable compound include epoxy compounds and oxetane compounds.

As the epoxy compound, for example, a glycidyl ether or the like can be used. Specific examples of the glycidyl ether include allyl glycidyl ether, phenyl glycidyl ether, 1,4-butanediol diglycidyl ether, 1,5-pentanediol diglycidyl ether, 1,6-hexanediol diglycidyl ether, 1,8-octanediol diglycidyl ether, 1,10-decanediol diglycidyl ether, 1,12-dodecanediol diglycidyl ether, ethylene glycol diglycidyl ether, diethylene glycol diglycidyl ether, polyethylene glycol diglycidyl ether, polypropylene glycol diglycidyl ether, neopentyl glycol diglycidyl ether, bisphenol A diglycidyl ether, bisphenol F diglycidyl ether, trimethylolpropane diglycidyl ether, glycerin triglycidyl ether, diglycerol triglycidyl ether, sorbitol polyglycidyl ether, pentaerythritol polyglycidyl ether, and the like, and one or more of these can be used.

Examples of the oxetane compound include 3-ethyl-3-hydroxymethyloxetane, 2-ethylhexyloxetane, xylylene bisoxetane, 3-ethyl-3{[(3-ethyloxetane-3-yl)methoxy]methyl}oxetane, 2-hydroxyethyl vinyl ether, 4-hydroxybutyl vinyl ether, diethylene glycol monovinyl ether, 2-ethylhexyl vinyl ether, and the like, and one or more of these can be used.

The chain transfer agent extracts a radical from the growth terminal of the polymerization reaction to stop the growth, and serves as a new polymerization reaction-initiating species to be add to the radical polymerizable monomer, and thus can start the growth of a new polymer. Use of the chain transfer agent increases the frequency of chain transfer of radical polymerization, so that the reaction rate of the radical polymerizable monomer increases, and the sensitivity to light can be improved. Furthermore, increase in the reaction rate of the radical polymerizable monomer increases components contributing to the reaction, and thus the degree of polymerization of the radical polymerizable monomer can be adjusted.

The chain transfer agent is not particularly limited, and examples of the chain transfer agent include α-methylstyrene dimer, 2-mercaptobenzoxazole, 2-mercaptobenzothiazole, tert-butyl alcohol, n-butanol, isobutanol, isopropylbenzene, ethylbenzene, chloroform, methyl ethyl ketone, propylene, vinyl chloride, and the like, and one or more of these can be used.

Examples of the polymerization inhibitor include quinone-based compounds such as hydroquinone and the like; hindered phenol-based compounds; benzotriazole compounds; thiazine-based compounds such as phenothiazine and the like; and the like, and one or more of these can be used.

As the UV sensitizer, for example, an anthracene-based compound or the like can be used.

[Influence of Oxygen Permeability of Base Material]

In the technology of the present application, the hologram recording medium 10 including the photosensitive layer 12 containing at least a polymerizable compound, a binder resin, and a photopolymerization initiator is selectively reacted using an electromagnetic ray having an amplitude modulated spatially. Furthermore, in the technology of the present application, first, the electromagnetic ray having an amplitude modulated spatially excites a sensitizing dye, and energy is transferred from the excited sensitizer to the photopolymerization initiator to excite the photopolymerization initiator. It is considered that radicals, hydrogen ions, and the like are further generated from the excited photopolymerization initiator and reacted with the polymerizable compound to form a diffraction grating while a growth terminal is generated and thus information is recorded. However, the above-described reaction proceeds even when a conventional hologram recording medium is light-shielded and stored in an unexposed state, and therefore it is considered that the material composition of the photosensitive layer 12 changes and thus the storage stability deteriorates.

Meanwhile, it is widely known that oxygen deactivates excited chemical species, and specifically, deactivation of an excited sensitizer or initiator and deactivation of a growth terminal in radical polymerization are known. In the hologram recording medium 10 according to the present embodiment, the oxygen permeability of the base material 11 is more than 0.1 cm³·(m²·day·atm)⁻¹, and thus the oxygen permeability is set to a higher value than in a conventional hologram recording medium. Therefore, it is considered that in the hologram recording medium 10 according to the present embodiment, oxygen is easily supplied to the photosensitive layer 12 and causes a deactivation reaction and therefore the material composition of the photosensitive layer 12 is less likely to change and thus deterioration of the storage stability is suppressed.

[Method of Manufacturing Hologram Recording Medium]

Hereinafter, an example of a method of manufacturing the hologram recording medium 10 according to the first embodiment will be described.

(Preparation of Photosensitive Composition)

Predetermined amounts of a compound represented by the general formula (1) described above, a photopolymerization initiator, and a binder resin are weighed out, added to a solvent at normal temperature or the like, and dissolved and mixed to prepare a photosensitive composition as a coating liquid. Furthermore, at least one selected from the group consisting of radical polymerizable monomers other than the compounds represented by the general formula (1) described above, sensitizing dyes, inorganic fine particles, plasticizers, chain transfer agents, polymerization inhibitors, UV sensitizers, and the like may be added according to the use, the purpose, and the like.

Examples of the solvent include acetone, xylene, toluene, methyl ethyl ketone, tetrahydrofuran, benzene, methylene chloride, dichloromethane, chloroform, methanol, ethanol, and the like, and one or more of these can be used. The solvent can be effective for adjusting the viscosity and the compatibility, and in addition, for improving the film-formability and the like.

(Step of Forming Photosensitive Layer)

The prepared photosensitive composition is applied onto a base material 11, and then dried to form a photosensitive layer 12. In the method of applying, for example, a spin coater, a gravure coater, a comma coater, a bar coater, or the like can be used. As described above, an intended hologram recording medium 10 can be obtained.

[Operation and Effect]

In the hologram recording medium 10 according to the first embodiment, the oxygen permeability of the base material 11 measured in an environment at 23° C. and 0% RH is more than 0.1 cm³·(m²·day·atm)⁻¹ and 10000 cm³·(m²·day·atm)⁻¹ or less, and the photosensitive layer 12 contains a compound represented by the general formula (1). As a result, the hologram recording medium 10 has a high diffraction efficiency and can achieve suppression of deterioration of the storage stability in an unexposed state.

[Modification]

(Modified Example 1)

As illustrated in FIG. 2, the hologram recording medium 10 may further include a release layer 13 on the second surface of the photosensitive layer 12. The release layer 13 is included so as to be peelable from the photosensitive layer 12. The release layer 13 may be a film or a substrate having rigidity, but is preferably a film from the viewpoint of increasing the oxygen permeability of the release layer 13. As the material of the release layer 13, a material similar to that of the base material 11 can be exemplified.

The lower limit of the oxygen permeability of the release layer 13 measured in an environment at 23° C. and 0% RH is more than 0.1 cm³·(m²·day·atm)⁻¹, preferably 15 cm³·(m²·day·atm)⁻¹ or more, more preferably 390 cm³·(m²·day·atm)⁻¹ or more, and still more preferably 590 cm³·(m²·day·atm)⁻¹ or more. If the lower limit of the oxygen permeability of the base material 11 is 0.1 cm³·(m²·day·atm)⁻¹ or more, deterioration of the storage stability in an unexposed state can be suppressed. The upper limit of the oxygen permeability of the release layer 13 measured in an environment at 23° C. and 0% RH is, for example, 10000 cm³·(m²·day·atm)⁻¹ or less, and preferably 2240 cm³·(m²·day·atm)⁻¹ or less. In a case where the lower limit of the oxygen permeability of the release layer 13 is more than 0.1 cm³·(m²·day·atm)⁻¹, the lower limit of the oxygen permeability of the base material 11 may be 0.1 cm³·(m²·day·atm)⁻¹ or less, or more than 0.1 cm³·(m²·day·atm)⁻¹.

The oxygen permeability of the release layer 13 is measured as follows. First, the release layer 13 is peeled off from the photosensitive layer 12. Next, the oxygen permeability of the peeled release layer 13 is measured. Details of the measurement conditions are similar to those in the method of measuring the oxygen permeability of the base material 11.

(Modified Example 2)

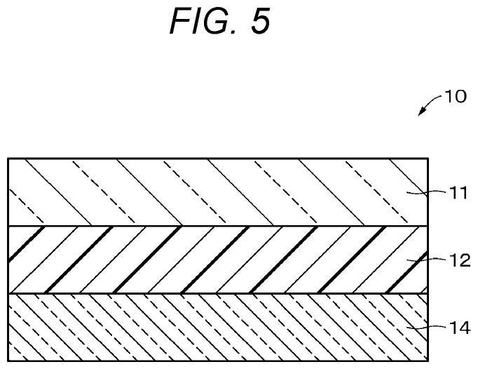

As illustrated in FIG. 5, the hologram recording medium 10 may further include a glass base material 14 on the second surface of the photosensitive layer 12. The glass base material 14 may be included so as to be peelable from the photosensitive layer 12. The glass base material 14 may be a glass substrate having rigidity or may be flexible glass.

### 3 Second Embodiment

[Configuration of Hologram Optical Element]

A hologram optical element according to a second embodiment of the present disclosure can be obtained by exposing a hologram recording medium 10. The hologram optical element includes an exposed photosensitive layer 12. The polymerizable compound contained in the exposed photosensitive layer 12 is polymerized. The exposed photosensitive layer 12 may contain, for example, a polymer and/or an oligomer containing a constituent unit derived from a compound represented by the general formula (1) and a binder resin described above and a photopolymerization initiator having a structure changed by irradiating the photopolymerization initiator with external energy to generate an active species.

The hologram optical element according to the second embodiment has a high diffraction efficiency and exhibits an effect of an excellent diffraction characteristic. Furthermore, the hologram optical element of the present embodiment is excellent in transparency.

[Optical System for Hologram Recording]

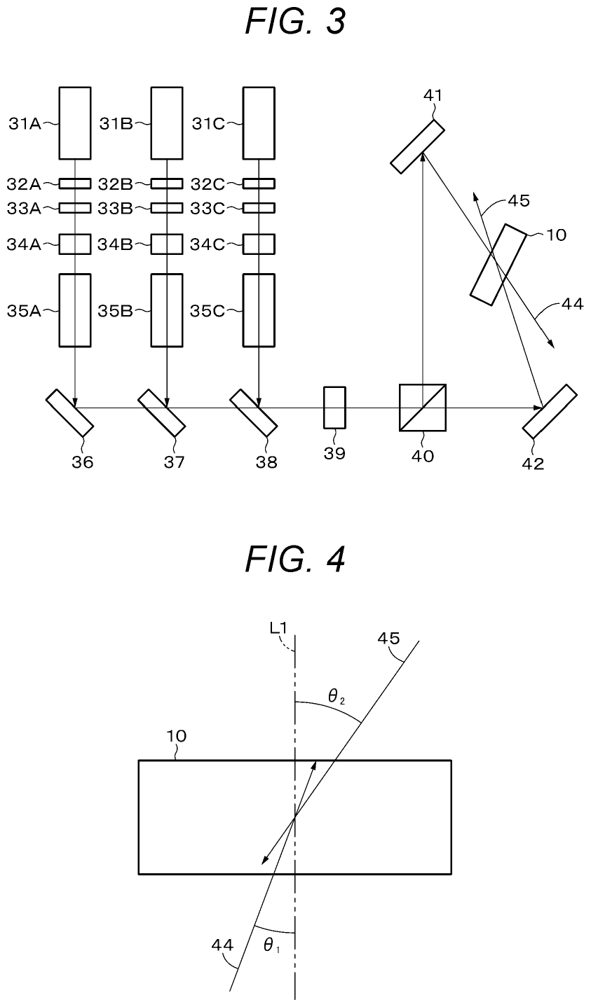

FIG. 3 is a schematic diagram illustrating an example of a configuration of an optical system for hologram recording used for exposure of the hologram recording medium 10. The optical system for hologram recording includes a diode-pumped solid-state laser 31A, a diode-pumped solid-state laser 31B, a diode-pumped solid-state laser 31C, electron shutters 32A, 32B, and 32C, half-wave plates 33A, 33B, and 33C, objective lenses 34A, 34B, and 34C, beam expanders 35A, 35B, and 35C, a mirror 36, a dichroic mirror 37, a dichroic mirror 38, an iris diaphragm 39, a beam splitter 40, a mirror 41, and a mirror 42.

The diode-pumped solid-state laser 31A emits red laser light having a peak wavelength of 660 nm. The red laser light emitted by the diode-pumped solid-state laser 31A is incident on the mirror 36 via the electron shutter 32A, the half-wave plate 33A, the objective lens 34A, and the beam expander 35A. The red laser light reflected by the mirror 36 is incident on the beam splitter 40 via the dichroic mirror 37, the dichroic mirror 38, and the iris diaphragm 39.

The diode-pumped solid-state laser 31B emits green laser light having a peak wavelength of 532 nm. The green laser light emitted by the diode-pumped solid-state laser 31B is incident on the dichroic mirror 37 via the electron shutter 32B, the half-wave plate 33B, the objective lens 34B, and the beam expander 35B. The dichroic mirror 37 reflects green laser light but transmits red laser light. The green laser light reflected by the dichroic mirror 37 is incident on the beam splitter 40 via the dichroic mirror 38 and the iris diaphragm 39.

The diode-pumped solid-state laser 31C emits blue laser light having a peak wavelength of 457 nm. The blue laser light emitted by the diode-pumped solid-state laser 31C is incident on the dichroic mirror 38 via the electron shutter 32C, the half-wave plate 33C, the objective lens 34C, and the beam expander 35C. The dichroic mirror 38 reflects blue laser light but transmits red laser light and green laser light. The blue laser light reflected by the dichroic mirror 38 is incident on the beam splitter 40 via the iris diaphragm 39.

Each color laser light incident on the beam splitter 40 is separated into a first light flux 44 and a second light flux 45. The separated first light flux 44 and second light flux 45 are reflected by the mirror 41 and the mirror 42, respectively, and thus the hologram recording medium 10 is irradiated with the first light flux 44 and the second light flux 45.

FIG. 4 is a schematic view of an optical system for hologram recording at the time of two-beam exposure. In the present description, an angle G₁ formed by the first light flux 44 and a normal line Li of the hologram recording medium 10 is defined as an “incident angle di of the first light flux 44”, and an angle θ₂ formed by the second light flux 45 and the normal line Li of the hologram recording medium 10 is defined as an “incident angle θ₂ of the second light flux 45”.

[Method of Manufacturing Hologram Optical Element]

The hologram optical element according to the second embodiment of the present disclosure can be obtained, for example, by performing two-beam exposure on the hologram recording medium 10 according to the first embodiment of the present disclosure using a semiconductor laser in the visible light region, then irradiating the entire surface with UV (ultraviolet ray) to cure an uncured monomer or the like, and fixing the refractive index distribution in the hologram recording medium 10. The conditions of the two-beam exposure may be appropriately set by those skilled in the art according to the use, the purpose, and the like of the hologram optical element, but the light intensity of one light flux on the hologram recording medium is preferably set to 0.1 to 100 mW/cm², exposure is preferably performed for 1 to 1000 seconds, and interference exposure is desirably performed so that the angle formed by two light fluxes is 0.1 to 179.9 degrees.

### 4 Third Embodiment

[Optical Device and Optical Component]

An optical device and an optical component of a third embodiment of the present disclosure include the hologram optical element according to the second embodiment of the present disclosure. Examples of the optical device and the optical component include image display devices such as eyewears, holographic screens, transparent displays, head-mounted displays, head-up displays, and the like, imaging devices, imaging elements, color filters, diffractive lenses, light-guiding plates, spectral elements, hologram sheets, information recording media such as optical disks, magneto-optical disks, and the like, optical pickup devices, polarizing microscopes, sensors, and the like.

The optical device and the optical component of the third embodiment according to the present disclosure use the hologram optical element excellent in a diffraction characteristic and transparency. Therefore, an optical device and an optical component can be realized that have a high optical characteristic and high optical stability. Moreover, in a case where the present disclosure is used in a display, the display can have a high see-through property.

### 5 Fourth Embodiment

[Method for Forming Hologram Diffraction Grating]

The present disclosure provides a method for forming a hologram diffraction grating, and the method includes selectively reacting, by using an electromagnetic ray having an amplitude modulated spatially, a hologram recording medium 10 including a photosensitive layer 12 containing a polymerizable compound containing a compound represented by the general formula (1) described above, a binder resin, and a photopolymerization initiator. The method for forming a hologram diffraction grating is the interference exposure described in the second embodiment. Therefore, description of the method for forming a hologram diffraction grating is omitted. According to the method for forming a hologram diffraction grating, an effect is exhibited as described in the third embodiment.

### 6 Analysis Method

[Method of Analyzing Onium Salt-Based Initiator, Substance Derived Therefrom, and the Like]

Examples of the method of analyzing an onium salt-based initiator and a substance derived from the onium salt-based initiator that are contained in a hologram recording medium 10 or a hologram optical element include, but are not limited to, the following analysis method 1 and analysis method 2.

<Analysis Method 1>

Analysis Method: LC-MS (Liquid Chromatography Mass Spectrometry)

Target substances for analysis: anionic components of onium salts, and decomposition products and reactants derived therefrom

Measurement sample: sample obtained by collecting photosensitive layer 12 from hologram recording medium 10, or by collecting photosensitive layer 12 after exposure from hologram optical element Note that details of the method of collecting these layers will be described later.

Details of measurement conditions of liquid chromatography (LC) and mass spectrometry (MS) are as follows.

<LC Measurement Conditions>

Liquid chromatography: Waters Aquity UPLC

Column: ACQUITY UPLC HSS-T3 (2.1 mm×100 mm, 1.8 μm)

Temperature: 40° C.

Flow rate: 0.3 mL/min

Mobile phase: A: 0.1% HCOOH aq. B: acetonitrile

Gradient: B: 40%-6 min-95% (6.8 min Hold)

Injection volume: Pos. 1.0 μL, Neg. 2.0 μL

<MS Measurement Conditions>

Mass spectrometer: Waters Synapt HDMS System

Measured mass range: m/z 100-1000

Ionization mode: ESI (−) (capillary voltage: −3.0 kV)

Ion source temperature: 120° C.

Heated desorption gas: N₂ 500° C. 800 L/hr

Cone voltage: 40 V

Collision energy: 20, 40, 60 eV

Mass resolution: 10,000 FWHM (V-mode) at m/z 556 (+)

Mass calibration substance: leucine enkephalin 100 ppb, at 50 μL/min

<Analysis Method 2>

Analysis Method: Py-GC/MS (Pyrolysis Gas Chromatograph Mass Spectrometry)

Analysis target substances: cationic components of onium salts, and decomposition products and reactants derived therefrom

Measurement sample: sample obtained by collecting photosensitive layer 12 from hologram recording medium 10, or by collecting photosensitive layer 12 after exposure from hologram optical element Note that details of the method of collecting these layers will be described later.

The analysis conditions are as follows.

Device: GC/MS HP6890+HP5973 (manufactured by Hewlett-Packard)

Double-Shot Pyrolyzer Py-2020D (manufactured by Frontier Laboratories Ltd.)

Heating temperature: 400° C.

Column: DB-5MS UI (0.25 mm×0.25 μm×30 m)

Injection method: split (split ratio 50:1)

Inlet temperature: 320° C.

Oven temperature: 50° C. (2 min)-20° C./min-320° C. (20 min)

Carrier gas: He (constant flow rate mode, 1.0 ml/min)

Mass range: m/z 29-700

[Method of Analyzing Organic Boron Salt-Based Initiator, Substance Derived Therefrom, and the Like]

Examples of the method of analyzing an organic boron salt-based initiator, a substance derived from the organic boron salt-based initiator, and the like contained in the hologram recording medium 10 or the hologram optical element also include, but are not limited to, the above-described analysis method 1 and analysis method 2.

[Method of Analyzing Compound Represented by General Formula (1), Polymer Thereof, and the Like]

Examples of the method of analyzing a compound represented by the general formula (1), a polymer of the compound, and the like contained in the hologram recording medium 10 or the hologram optical element include, but are not limited to, the following analysis method.

<Analysis Method>

Analysis Method: Py-GC/MS (Pyrolysis Gas Chromatograph Mass Spectrometry)

Analysis target substance: compound represented by general formula (1), or compound having same mother skeleton as that in general formula (1)

Measurement sample: sample obtained by collecting photosensitive layer 12 from hologram recording medium 10, or by collecting photosensitive layer 12 after exposure from hologram optical element Note that details of the method of collecting these layers will be described later.

Device: GC/MS HP6890+HP5973 (manufactured by Hewlett-Packard)

Double-Shot Pyrolyzer Py-2020D (manufactured by Frontier Laboratories Ltd.)

Details of the analysis conditions are as follows.

Heating temperature: 400° C.

Column: DB-5MS UI (0.25 mm×0.25 μm×30 m)

Injection method: split (split ratio 50:1)

Inlet temperature: 320° C.

Oven temperature: 50° C. (2 min)-20° C./min-320° C. (20 min)

Carrier gas: He (constant flow rate mode, 1.0 ml/min)

Mass range: m/z 29-700

[Method of Collecting Photosensitive Layer 12 from Hologram Recording Medium 10]

Examples of the method of collecting the photosensitive layer 12 from the hologram recording medium 10 include, but are not limited to, the following method. First, in a case where a release layer 13 is attached, the release layer 13 is peeled off from the photosensitive layer 12. Subsequently, the photosensitive layer 12 is rubbed off from a base material 11, and thus the photosensitive layer 12 is collected. Alternatively, the photosensitive layer 12 with the base material 11 still attached is immersed in an organic solvent that does not affect the base material 11 to extract the components of the photosensitive layer 12 into the organic solvent. Even in a case where the release layer 13 cannot be peeled off from the photosensitive layer 12, extraction with an organic solvent can be used. As the organic solvent that does not affect the base material 11 and the release layer 13, for example, an organic solvent is used such as acetone, methyl ethyl ketone, methanol, ethanol, tetrahydrofuran, toluene, methylene chloride, chloroform, or the like.

[Method of Collecting Photosensitive Layer 12 after Exposure from Hologram Optical Element]

Examples of the method of collecting a photosensitive layer 12 after exposure from a hologram recording medium 20 include, but are not limited to, the following method. First, in a case where a support is attached, the support is peeled off from the photosensitive layer 12 after exposure. Subsequently, the photosensitive layer 12 after exposure is rubbed off from a base material 11, and thus the photosensitive layer 12 after exposure is collected. Alternatively, the photosensitive layer 12 after exposure with the base material 11 still attached is immersed in an organic solvent that does not affect the base material 11 to extract the components of the photosensitive layer 12 after exposure into the organic solvent. Even in a case where the support cannot be peeled off from the photosensitive layer 12 after exposure, extraction with an organic solvent can be used. As the organic solvent that does not affect the base material 11 and the support, for example, an organic solvent is used such as acetone, methyl ethyl ketone, methanol, ethanol, tetrahydrofuran, toluene, methylene chloride, chloroform, or the like.

### EXAMPLES

Hereinafter, the present disclosure will be specifically described with reference to examples, but the present disclosure is not limited to only these examples.

In the following Examples and Comparative Examples, the oxygen permeability of a film as a base material is the oxygen permeability of the base material measured in an environment at 23° C. and 0% RH, and is a value determined with the measurement method described in the first embodiment.

[Synthesis of Polymerizable Compound]

The polymerizable compounds (compounds represented by the chemical formula (6-3) and the chemical formula (11-10), respectively) used in the following Examples and Comparative Examples were synthesized as follows.

### Test Example 1

(Synthesis Method of Compound Represented by Chemical Formula (6-3))

The synthesis method (synthesis route) of the compound represented by the chemical formula (6-3) is as follows.

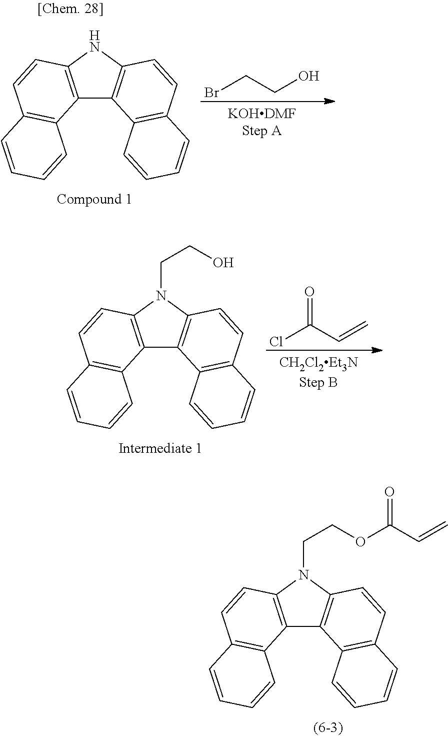

(Step A)

The step A in the synthesis route shown above will be described.

Under an inert atmosphere, 110 mL of an N,N-dimethylformamide (manufactured by KANTO CHEMICAL CO., INC.) solution was prepared by mixing with 20 g of potassium hydroxide (manufactured by KANTO CHEMICAL CO., INC.), 15 g of a compound 1 (7H-dibenzo[c,g]carbazole (manufactured by Tokyo Chemical Industry Co., Ltd.)) was added, the resulting mixture was stirred for 1 hour, and then 25 g of 2-bromoethanol (manufactured by Tokyo Chemical Industry Co., Ltd.) was added and reacted for 20 hours. The resulting product was quenched by adding water, and subjected to extraction with toluene using a separatory funnel and to column purification, and thus 10 g of a target product (intermediate 1) was obtained.

(Step B)

The step B in the synthesis route shown above will be described.

In a solution obtained by mixing triethylamine (manufactured by KANTO CHEMICAL CO., INC.) with 50 mL of methylene chloride (manufactured by KANTO CHEMICAL CO., INC.), 9 g of the intermediate 1 was dissolved, and the solution was cooled in an ice bath. Thereafter, 3 mL of acryloyl chloride (manufactured by Tokyo Chemical Industry Co., Ltd.) was added little by little, and the mixture was naturally heated to room temperature and reacted for 4 hours. The resulting product was quenched by adding water and subjected to extraction with methylene chloride (manufactured by KANTO CHEMICAL CO., INC.) using a separatory funnel, then the organic layer was washed with saline and subjected to silica filtration and then to column purification to obtain 6 g of a compound of Test Example 1 (compound represented by the chemical formula (6-3)).

The structure of the compound of Test Example 1 (compound represented by the chemical formula (6-3)) was identified using NMR. The result of NMR is as follows.

1H NMR (CDCl₃): 4.60-4.64 (2H), 4.85-4.89 (2H), 5.74-5.76 (1H), 5.95-6.05 (1H), 6.25-6.31 (1H), 7.49-7.55 (2H), 7.65-7.69 (2H), 7.70-7.77 (2H), 7.91-7.94 (2H), 8.03-8.06 (2H), 9.18-9.22 (2H)

### Test Example 2

(Synthesis Method of Compound Represented by Chemical Formula (11-10))

The synthesis method (synthesis route) of the compound represented by the chemical formula (11-10) is as follows.

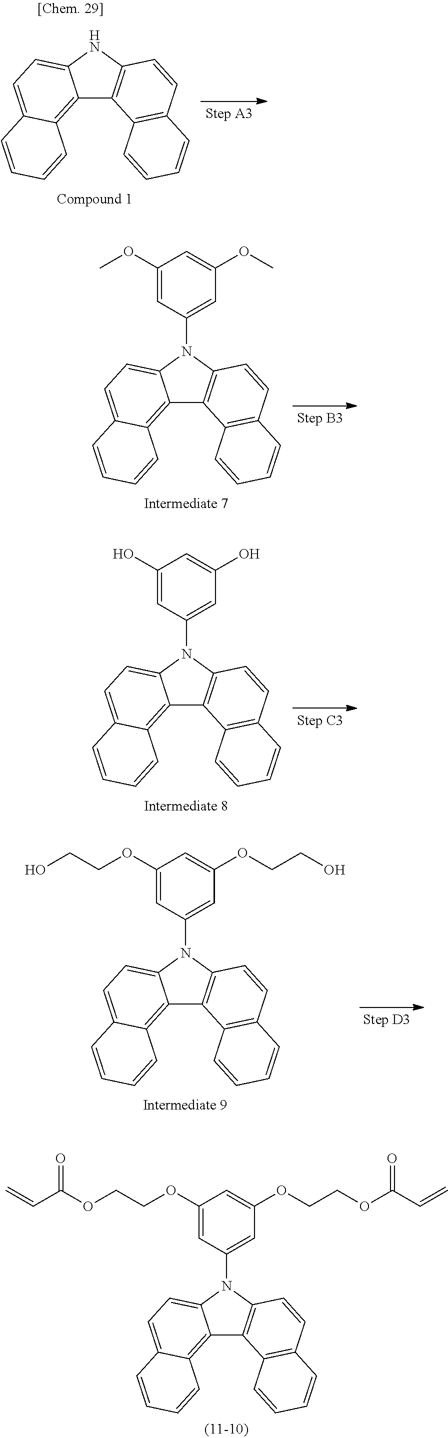 [SMILES: c1ccc2c(c1)ccc1[nH]c3ccc4ccccc4c3c12 ; COc1cc(OC)cc(-n2c3ccc4ccccc4c3c3c4ccccc4ccc32)c1 ; Oc1cc(O)cc(-n2c3ccc4ccccc4c3c3c4ccccc4ccc32)c1 ; OCCOc1cc(OCCO)cc(-n2c3ccc4ccccc4c3c3c4ccccc4ccc32)c1 ; C=CC(=O)OCCOc1cc(OCCOC(=O)C=C)cc(-n2c3ccc4ccccc4c3c3c4ccccc4ccc32)c1]

(Step A3)

The step A3 in the synthesis route shown above will be described.

Under an inert atmosphere, 90 g of the compound 1 (7H-dibenzo[c,g]carbazole (manufactured by Tokyo Chemical Industry Co., Ltd.)), 146 g of 1-bromo-3,5-dimethoxybenzene (manufactured by Tokyo Chemical Industry Co., Ltd.), and 214 g of tripotassium phosphate (manufactured by FUJIFILM Wako Pure Chemical Corporation) were dissolved in 1.8 L of deoxidized toluene (manufactured by FUJIFILM Wako Pure Chemical Corporation), then 80 mL of 1,2-cyclohexanediamine (manufactured by Tokyo Chemical Industry Co., Ltd.) and 64 g of copper (I) iodide (manufactured by FUJIFILM Wako Pure Chemical Corporation) were added, and the resulting mixture was stirred under heating and refluxing for 2 days. After allowing the resulting reaction liquid to cool to room temperature, the reaction liquid was filtered, 141 g of Si-Thiol (manufactured by Biotage Japan Ltd.) was added to the filtrate, the resulting mixture was stirred for 30 minutes, and then Si-Thiol was removed by filtration to obtain 63 g of a target product (intermediate 7).

(Step B3)

The step B3 in the synthesis route shown above will be described.

Under an inert atmosphere, 62 g of the intermediate 7 was dissolved in 870 mL of super dehydrated chloroform (product with added amylene, manufactured by FUJIFILM Wako Pure Chemical Corporation), then 700 mL of a boron tribromide-dichloromethane solution (concentration: 1.0 mol/L, manufactured by FUJIFILM Wako Pure Chemical Corporation) after ice cooling was added dropwise, and the mixture was stirred under ice cooling for 2.5 hours, then returned to room temperature, and further stirred for 2.5 hours. After further ice cooling, 200 mL of a boron tribromide-dichloromethane solution (concentration: 1.0 mol/L) was added dropwise, the mixture was heated to room temperature and stirred, then the reaction liquid was poured into ice water and thus quenched, washed with chloroform, subjected to recrystallization in a refrigerator, and filtered, and then the filtrate was subjected to column purification to obtain 60 g of a target product (intermediate 8).

(Step C3)

The step C3 in the synthesis route shown above will be described.

Under an inert atmosphere, 230 mL of an aqueous sodium hydroxide solution (concentration: 8.0 mol/L, manufactured by FUJIFILM Wako Pure Chemical Corporation), 8.5 g of tetrabutylammonium bromide (manufactured by Tokyo Chemical Industry Co., Ltd.), and 25 g of the intermediate 8 were mixed and cooled with ice water, and then 46 mL of 2-(2-chloroethoxy)tetrahydro-2H-pyran (manufactured by Tokyo Chemical Industry Co., Ltd.) was added. The resulting mixture was stirred at 80° C. and then allowed to cool to room temperature, and water and chloroform (manufactured by Tokyo Chemical Industry Co., Ltd.) were added to perform liquid separation. The obtained organic layer was subjected to concentration, drying, and the like to obtain 66 g of a brown oil. The obtained oil was subjected to column purification and the like to obtain 17 g of a target product (intermediate 9).

(Step D3)

The step D3 in the synthesis route shown above will be described.

To 50 mL of super dehydrated tetrahydrofuran (manufactured by FUJIFILM Wako Pure Chemical Corporation), 10 mg of phenothiazine (manufactured by Tokyo Chemical Industry Co., Ltd.) was added to produce a phenothiazine preparation liquid. Under an inert atmosphere, 16 g of the intermediate 9 and 5.0 mL of the phenothiazine preparation liquid were mixed, and 0.87 g of 4-dimethylaminopyridine (manufactured by Tokyo Chemical Industry Co., Ltd.) and 2.4 mL of acrylic acid (manufactured by Tokyo Chemical Industry Co., Ltd.) were added. After cooling with ice water, 15 mL of N,N′-diisopropylcarbodiimide (manufactured by FUJIFILM Wako Pure Chemical Corporation) was added, and the mixture was returned to room temperature and stirred overnight. The reaction liquid was filtered, and 210 mL of super dehydrated tetrahydrofuran, 1.1 g of 4-dimethylaminopyridine, and 7.3 mL of acrylic acid were mixed with the obtained filtrate. After cooling with ice water, 19 mL of N,N′-diisopropylcarbodiimide was added, and then the mixture was returned to room temperature and stirred overnight. The reaction liquid was filtered, and the filtrate was concentrated under reduced pressure and then subjected to column purification, 5.0 mL of the phenothiazine preparation liquid was added to the obtained fraction, and the resulting mixture was concentrated and dried to obtain 5.9 g of a compound of Test Example 2 (compound represented by the chemical formula (11-10)).

The structure of the compound of Test Example 2 (compound represented by the chemical formula (11-10)) was identified using NMR. The result of NMR is as follows.

1H NMR (CDCl₃): 4.24-4.26 (4H), 4.52-4.56 (4H), 5.84-5.88 (2H), 6.12-6.21 (2H), 6.42-6.49 (2H), 6.71-6.78 (3H), 7.51-8.05 (10H), 9.23-9.25 (2H)

### Example 1-1

(Step of Preparing Photosensitive Composition)

The radical polymerizable monomers, the binder resin, the plasticizer, the sensitizing dye, the onium salt-based initiators, the organic boron salt-based initiator, the chain transfer agent, the polymerization inhibitor, and the UV sensitizer described in Table 1 were prepared, and these were weighed out at a proportion described in Table 1 and mixed in a solvent at normal temperature to prepare a photosensitive composition. As the solvent, a mixture obtained by mixing methyl ethyl ketone and ethanol in advance at a weight ratio of 80 and 20, respectively, was used.

(Step of Producing Hologram Recording Medium)

The photosensitive composition was applied, with a bar coater so as to have a dry film thickness of 5 μm, onto a polyethylene terephthalate film (hereinafter, referred to as “PET film”) having a thickness of 100 μm, and then the thin film surface of the photosensitive layer including the photosensitive composition was pressure-bonded onto a glass substrate having a thickness of 1.0 mm to produce a hologram recording medium.

### Example 1-2

A hologram recording medium was produced in a manner similar to that in Example 1-1 except that a polycarbonate film with a hard coat (hereinafter, referred to as “HC-PC film”) (total thickness: 110 μm) was used in place of the PET film having a thickness of 100 μm.

### Examples 1-3 to 1-5

A hologram recording medium was produced in a manner similar to that in Example 1-1 except that a cycloolefin polymer film (hereinafter, referred to as “COP film”) having a thickness of 50 μm (Example 1-3), 23 μm (Example 1-4), or 13 μm (Example 1-5) was used in place of the PET film having a thickness of 100 μm.

### Examples 1-6, 1-8, 1-10, and 1-12

Irgacure 290 (Example 1-6), T2041 (Example 1-8), CPI-100P (Example 1-10), and CPI-310B (Example 1-12) were used in place of 10591 as an onium salt used in Example 1-1, the radical polymerizable monomers, the binder resin, the plasticizer, the sensitizing dye, the onium salt-based initiators, the organic boron salt-based initiator, the chain transfer agent, the polymerization inhibitor, and the UV sensitizer described in Table 2 were prepared, and these were weighed out at a proportion described in Table 2 and mixed in a solvent at normal temperature to prepare a photosensitive composition. As the solvent, a mixture obtained by mixing methyl ethyl ketone and ethanol in advance at a weight ratio of 80 and 20, respectively, was used.

Moreover, each photosensitive composition described above was applied, with a bar coater so as to have a dry film thickness of 5 μm, onto a COP film having a thickness of 50 μm, and the thin film surface of the photosensitive layer including the photosensitive composition was pressure-bonded onto a glass substrate having a thickness of 1.0 mm to produce a hologram recording medium.

### Example 1-7

The photosensitive composition prepared in Example 1-6 was applied, with a bar coater so as to have a dry film thickness of 5 μm, onto a COP film having a thickness of 50 μm, the thin film surface of the photosensitive layer including the photosensitive composition was pressure-bonded onto a release-treated surface of a PET film having a thickness of 100 μm to produce a film-shaped hologram recording medium, and the film-shaped hologram recording medium was stored in a light-shielding state in a double black bag. After the storage in a light-shielding state, the PET film was peeled off from the film-shaped hologram recording medium immediately before formation of a hologram diffraction grating, and the thin film surface of the photosensitive layer including the photosensitive composition was pressure-bonded onto a glass substrate having a thickness of 1.0 mm to produce a hologram recording medium.

### Example 1-9

The photosensitive composition prepared in Example 1-8 was applied, with a bar coater so as to have a dry film thickness of 5 μm, onto a COP film having a thickness of 50 μm, the thin film surface of the photosensitive layer including the photosensitive composition was pressure-bonded onto a release-treated surface of a PET film having a thickness of 100 μm to produce a film-shaped hologram recording medium, and the film-shaped hologram recording medium was stored in a light-shielding state in a double black bag. After the storage in a light-shielding state, the PET film was peeled off from the film-shaped hologram recording medium immediately before formation of a hologram diffraction grating, and the thin film surface of the photosensitive layer including the photosensitive composition was pressure-bonded onto a glass substrate having a thickness of 1.0 mm to produce a hologram recording medium.

### Example 1-11

The photosensitive composition prepared in Example 1-10 was applied, with a bar coater so as to have a dry film thickness of 5 μm, onto a COP film having a thickness of 50 μm, the thin film surface of the photosensitive layer including the photosensitive composition was pressure-bonded onto a release-treated surface of a PET film having a thickness of 100 μm to produce a film-shaped hologram recording medium, and the film-shaped hologram recording medium was stored in a light-shielding state in a double black bag. After the storage in a light-shielding state, the PET film was peeled off from the film-shaped hologram recording medium immediately before formation of a hologram diffraction grating, and the thin film surface of the photosensitive layer including the photosensitive composition was pressure-bonded onto a glass substrate having a thickness of 1.0 mm to produce a hologram recording medium.

### Example 1-13

The photosensitive composition prepared in Example 1-12 was applied, with a bar coater so as to have a dry film thickness of 5 μm, onto a COP film having a thickness of 50 μm, the thin film surface of the photosensitive layer including the photosensitive composition was pressure-bonded onto a release-treated surface of a PET film having a thickness of 100 μm to produce a film-shaped hologram recording medium, and the film-shaped hologram recording medium was stored in a light-shielding state in a double black bag. After the storage in a light-shielding state, the PET film was peeled off from the film-shaped hologram recording medium immediately before formation of a hologram diffraction grating, and the thin film surface of the photosensitive layer including the photosensitive composition was pressure-bonded onto a glass substrate having a thickness of 1.0 mm to produce a hologram recording medium.

### Comparative Example 1

A hologram recording medium was produced in a manner similar to that in Example 1-1 except that a laminated film obtained by stacking polyvinyl alcohol having a thickness of 2.5 μm and a cycloolefin polymer film having a thickness of 50 μm (hereinafter, referred to as “PVA/COP film”) was used in place of the PET film having a thickness of 100 μm.

### Examples 2-1 and 2-2

The radical polymerizable monomers, the binder resin, the plasticizer, the sensitizing dye, the onium salt-based initiators, the organic boron salt-based initiator, the chain transfer agent, the polymerization inhibitor, and the UV sensitizer described in Table 3 were prepared, and these were weighed out at a proportion described in Table 3. Furthermore, in Example 2-1, a PET film having a thickness of 100 μm was used, and in Example 2-2, the PET film having a thickness of 100 μm was replaced with a COP film having a thickness of 50 μm. A hologram recording medium was produced in a manner similar to that in Example 1-1 except for these procedures.

### Comparative Example 2

The PET film having a thickness of 100 μm was replaced with a PVA/COP film obtained by stacking a PVA film having a thickness of 2.5 μm and a COP film having a thickness of 50 μm. A hologram recording medium was produced in a manner similar to that in Example 2-1 except for this procedure.

### Examples 3-1 and 3-2

The radical polymerizable monomers, the binder resin, the plasticizer, the sensitizing dye, the onium salt-based initiators, the organic boron salt-based initiator, the chain transfer agent, the polymerization inhibitor, and the UV sensitizer described in Table 3 were prepared, and these were weighed out at a proportion described in Table 3. Furthermore, in Example 3-1, a PET film having a thickness of 100 μm was used, and in Example 3-2, a COP film having a thickness of 50 μm was used. A hologram recording medium was produced in a manner similar to that in Example 2-1 except for these procedures.

### Comparative Example 3

The PET film having a thickness of 100 μm was replaced with a PVA/COP film obtained by stacking a PVA film having a thickness of 2.5 μm and a COP film having a thickness of 50 μm. A hologram recording medium was produced in a manner similar to that in Example 3-1 except for this procedure.

### Examples 4-1 and 4-2

The radical polymerizable monomers, the binder resin, the plasticizer, the sensitizing dye, the onium salt-based initiators, the organic boron salt-based initiator, the chain transfer agent, the polymerization inhibitor, and the UV sensitizer described in Table 3 were prepared, and these were weighed out at a proportion described in Table 3. Furthermore, in Example 4-1, a PET film having a thickness of 100 μm was used, and in Example 4-2, a COP film having a thickness of 50 μm was used in place of the PET film having a thickness of 100 μm. A hologram recording medium was produced in a manner similar to that in Example 2-1 except for these procedures.

### Comparative Example 4

The PET film having a thickness of 100 μm was replaced with a PVA/COP film obtained by stacking a PVA film having a thickness of 2.5 μm and a COP film having a thickness of 50 μm. A hologram recording medium was produced in a manner similar to that in Example 3-1 except for this procedure.

### Examples 5-1 and 5-2

Irgacure 290 was used in place of 10591 as an onium salt used in Example 1-1, the radical polymerizable monomers, the binder resin, the plasticizers, the sensitizing dyes, the onium salt-based initiators, the organic boron salt-based initiator, the chain transfer agent, the polymerization inhibitor, the UV sensitizer, and the color former described in Table 4 were prepared, and these were weighed out at a proportion described in Table 4 and mixed in a solvent at normal temperature to prepare a photosensitive composition. As the solvent, a mixture obtained by mixing methyl ethyl ketone and ethanol in advance at a weight ratio of 80 and 20, respectively, was used.

Moreover, each photosensitive composition described above was applied, with a bar coater so as to have a dry film thickness of 5 μm, onto an HC-PC film having a thickness of 110 μm (Example 5-1) or a COP film having a thickness of 50 μm (Example 5-2), and the thin film surface of the photosensitive layer including the photosensitive composition was pressure-bonded onto a glass substrate having a thickness of 1.0 mm to produce a hologram recording medium.

### Examples 5-3 to 5-5

The photosensitive composition prepared in Example 5-1 was applied, with a bar coater so as to have a dry film thickness of 5 μm, onto each of an HC-PC film having a thickness of 110 μm (Example 5-3), a COP film having a thickness of 50 μm (Example 5-4), and a PVA/COP film obtained by stacking a PVA film having a thickness of 2.5 μm and a COP film having a thickness of 50 μm (Example 5-5), the thin film surface of the photosensitive layer including the photosensitive composition was pressure-bonded onto a release-treated surface of a PET film having a thickness of 100 μm to produce a film-shaped hologram recording medium, and the film-shaped hologram recording medium was stored in a light-shielding state in a double black bag. After the storage in a light-shielding state, the PET film was peeled off from the film-shaped hologram recording medium immediately before formation of a hologram diffraction grating, and the thin film surface of the photosensitive layer including the photosensitive composition was pressure-bonded onto a glass substrate having a thickness of 1.0 mm to produce a hologram recording medium.

### Example 5-6

In a manner similar to that in Example 1-1, the radical polymerizable monomers, the binder resin, the plasticizers, the sensitizing dyes, the onium salt-based initiators, the organic boron salt-based initiator, the chain transfer agent, the polymerization inhibitor, the UV sensitizer, and the color former described in Table 4 were prepared, and these were weighed out at a proportion described in Table 4 and mixed in a solvent at normal temperature to prepare a photosensitive composition. As the solvent, a mixture obtained by mixing methyl ethyl ketone and ethanol in advance at a weight ratio of 80 and 20, respectively, was used.

Moreover, the photosensitive composition described above was applied, with a bar coater so as to have a dry film thickness of 5 μm, onto a COP film having a thickness of 50 μm, and the thin film surface of the photosensitive layer including the photosensitive composition was pressure-bonded onto a glass substrate having a thickness of 1.0 mm to produce a hologram recording medium.

### Examples 5-7 and 5-8

The photosensitive composition prepared in Example 5-6 was applied, with a bar coater so as to have a dry film thickness of 5 μm, onto each of a COP film having a thickness of 50 μm (Example 5-7) and a PVA/COP film obtained by stacking a PVA film having a thickness of 2.5 μm and a COP film having a thickness of 50 μm (Example 5-8), the thin film surface of the photosensitive layer including the photosensitive composition was pressure-bonded onto a release-treated surface of a PET film having a thickness of 100 μm to produce a film-shaped hologram recording medium, and the film-shaped hologram recording medium was stored in a light-shielding state in a double black bag. After the storage in a light-shielding state, the PET film was peeled off from the film-shaped hologram recording medium immediately before formation of a hologram diffraction grating, and the thin film surface of the photosensitive layer including the photosensitive composition was pressure-bonded onto a glass substrate having a thickness of 1.0 mm to produce a hologram recording medium.

### Comparative Example 5

The photosensitive composition prepared in Example 5-6 was applied, with a bar coater so as to have a dry film thickness of 5 μm, onto a PVA/COP film obtained by stacking a PVA film having a thickness of 2.5 μm and a COP film having a thickness of 50 μm, and the thin film surface of the photosensitive layer including the photosensitive composition was pressure-bonded onto a glass substrate having a thickness of 1.0 mm to produce a hologram recording medium.

[Evaluation of Characteristics of Hologram]

First, the hologram recording medium immediately after the production was subjected to two-beam interference exposure using a diode-pumped solid-state laser having exposure wavelengths of 457 nm, 532 nm, and 660 nm. Thereafter, the entire surface was irradiated with UV light to cure the uncured monomer, and the refractive index distribution was fixed in the hologram recording medium. As a result, a hologram optical element was obtained. Subsequently, the diffraction efficiency [%] of the hologram optical element was determined with the following evaluation method. Tables 1, 2, 3, and 4 show the results. Hereinafter, this diffraction efficiency may be referred to as diffraction efficiency of the initial hologram optical element.

(Evaluation of Diffraction Efficiency)

In the hologram optical element, the transmitted light intensity of an area not subjected to two-beam interference exposure was measured using a spectroscope and a light source that emits light having a wavelength of at least 400 to 700 nm, and the obtained value was defined as reference light intensity. The transmitted light intensity of the area in which the hologram optical element was subjected to two-beam interference exposure was measured using a similar device, and the obtained value was taken as the sample light intensity. In the case of measuring the sample light intensity and the reference light intensity, the incident angle from the light source to the hologram optical element was in accordance with the incident angle of the first light flux or the second light flux at the time of hologram recording, and the light incident on the hologram optical element for determining the sample light intensity was defined as reproduction illumination light.

The diffraction efficiency (%) was determined from the following formula.

Diffraction efficiency (%); 100−(sample light intensity at each wavelength)/(transmitted light intensity at each wavelength)×100

Furthermore, the following devices were used as the diode-pumped solid-state laser and the UV light irradiation device, and for measurement of the diffraction efficiency.

<Diode-Pumped Solid-State Laser>

660 nm Diode-pumped solid-state laser: manufactured by Cobolt AB, product name: Flamenco 660 nm

532 nm Diode-pumped solid-state laser: manufactured by Cobolt AB, product name: Samba 532 nm

457 nm Diode-pumped solid-state laser: manufactured by Melles Griot, product name: 85 BLS 601 457 nm

<UV Light Irradiation Device>

CV-1LC-G: manufactured by Heraeus, product name: CV-1LC-G, LED lamp used: Semray UV4003, integrated UV light amount: 4 J-cm-2, wavelength: 365 nm)<Device for Measurement of Diffraction Efficiency>

Light source: manufactured by MATSU PHOTONICS K.K., product name: LIGHTNINGCURE, spot light source LC8

Spectrometer: manufactured by Ocean Optics, Inc., product name: USB4000 Miniature Fiber Optic Spectrometer

Note that the maximum diffraction efficiency used was the highest diffraction efficiency of the diffraction efficiencies at wavelengths obtained by the method described in (Evaluation of Diffraction Efficiency) above. In addition, the grating period and the grating inclination angle of the diffraction grating were determined on the basis of the description in Bell Syst. Tech. J., 48, 2909 (1969) from the incident angles of the first light flux and the second light flux at the time of hologram recording, the wavelengths of the first light flux and the second light flux, and the average refractive index of the photosensitive layer of the hologram recording medium.

[Evaluation of Storage Stability]

First, the hologram recording medium immediately after the production was put in a double black bag and stored in a light-shielding state for one week, then the hologram recording medium was taken out, and the diffraction efficiency [%] of the hologram optical element after the storage for one week was determined with a procedure similar to that in the evaluation of the characteristics of the hologram described above. Next, the retention [%] was determined as shown in (1) or (2) described below according to the number of sensitizing dyes used in the photosensitive composition. Tables 1, 2, 3, and 4 show the results.

(1) Retention in the case of using one sensitizing dye

Retention [%]=((diffraction efficiency of hologram optical element after storage for one week)/(diffraction efficiency of initial hologram optical element))×100

(2) Retention in the case of using two or more sensitizing dyes

Retention [%]=((total of diffraction efficiency of hologram optical element after storage for one week)/(total of diffraction efficiency of initial hologram optical element))×100

TABLE 1

| | | | Example | Example | Example | Example | Example | Comparative |
|---|---|---|---|---|---|---|---|---|
| | | | 1-1 | 1-2 | 1-3 | 1-4 | 1-5 | Example 1 |
| Photosensitive | Monomer | EA-0200 | 33.85 | | | | | |
| composition | | EACz | 7.54 | | | | | |
| | Binder resin | SN-55T | 17.82 | | | | | |
| | Plasticizer | SDE | 27.59 | | | | | |
| | Sensitizing dye | MB | 0.43 | | | | | |
| | Initiator | I0591 | 7.75 | | | | | |
| | | (onium salt- | | | | | | |
| | | based initiator) | | | | | | |
| | | Irgacure 290 | — | | | | | |
| | | (onium salt- | | | | | | |
| | | based initiator) | | | | | | |
| | | T2041 | — | | | | | |
| | | (onium salt- | | | | | | |
| | | based initiator) | | | | | | |
| | | CPI-100P | — | | | | | |
| | | (onium salt- | | | | | | |
| | | based initiator) | | | | | | |
| | | CPI-310B | — | | | | | |
| | | (onium salt- | | | | | | |
| | | based initiator) | | | | | | |
| | | P3B | 3.15 | | | | | |
| | | (organic boron | | | | | | |
| | | salt-based | | | | | | |
| | | initiator) | | | | | | |
| | Chain transfer agent | 2-MBO | 0.52 | | | | | |
| | Polymerization | PT | 0.07 | | | | | |
| | inhibitor | | | | | | | |
| | UV sensitizer | UVS-1331 | 1.28 | | | | | |
| | Solid concentration | | 27.6 | | | | | |
| Base material (1) | Material | | PET | HC-PC | COP | COP | COP | PVA/COP |
| | Thickness [μm] | | 100 | 110 | 50 | 23 | 13 | 52.5 |
| | Oxygen permeability | | 15 | 390 | 590 | 1300 | 2240 | 0.1 |
| | [cm³ · (m² · day · atm)⁻¹] | | | | | | | |
| Base material (2) | Material | | Glass | | | | | |
| | Thickness [μm] | | 1000 | | | | | |
| | Oxygen permeability | | — | | | | | |
| | [cm³ · (m² · day · atm)⁻¹] | | | | | | | |
| Exposure condition | Incident angle θ₁ of first light flux [deg] | | 6.5 | | | | | |
| | Incident angle θ₂ of second light flux [deg] | | 9.5 | | | | | |
| Evaluation result | Hologram | Diffraction | 40 | 41 | 42 | 44 | 40 | 40 |
| | characteristic | efficiency [%] | | | | | | |
| | Storage stability | Retention [%] | 62 | 88 | 94 | 81 | 102 | 0 |

TABLE 2

| | | | Example | Example | Example | Example |
|---|---|---|---|---|---|---|
| | | | 1-6 | 1-7 | 1-8 | 1-9 |
| Photosensitive | Monomer | EA-0200 | 32.90 | | 35.37 | |
| composition | | EACz | 7.33 | | 7.88 | |
| | Binder resin | SN-55T | 17.33 | | 18.63 | |
| | Plasticizer | SDE | 26.82 | | 28.84 | |
| | Sensitizing dye | MB | 0.42 | | 0.45 | |
| | Initiator | I0591 | — | | — | |
| | | (onium salt- | | | | |
| | | based | | | | |
| | | initiator) | | | | |
| | | Irgacure 290 | 10.33 | | — | |
| | | (onium salt- | | | | |
| | | based | | | | |
| | | initiator) | | | | |
| | | T2041 | — | | 3.59 | |
| | | (onium salt- | | | | |
| | | based | | | | |
| | | initiator) | | | | |
| | | CPI-100P | — | | — | |
| | | (onium salt- | | | | |
| | | based | | | | |
| | | initiator) | | | | |
| | | CPI-310B | — | | — | |
| | | (onium salt- | | | | |
| | | based | | | | |
| | | initiator) | | | | |
| | | P3B | 3.06 | | 3.29 | |
| | | (organic boron | | | | |
| | | salt-based | | | | |
| | | initiator) | | | | |
| | Chain transfer | 2-MBO | 0.50 | | 0.54 | |
| | agent | | | | | |
| | Polymerization | PT | 0.07 | | 0.08 | |
| | inhibitor | | | | | |
| | UV sensitizer | UVS-1331 | 1.24 | | 1.34 | |
| | Solid concentration | | 27.6 | | | |
| Base material (1) | Material | | COP | COP | COP | COP |
| | Thickness [μm] | | 50 | 50 | 50 | 50 |
| | Oxygen permeability | | 590 | 590 | 590 | 590 |
| | [cm³ · (m² · day · atm)⁻¹] | | | | | |
| Base material (2) | Material | | Glass | PET | Glass | PET |
| | Thickness [μm] | | 1000 | 100 | 1000 | 100 |
| | Oxygen permeability | | — | 15 | — | 15 |
| | [cm³ · (m² · day · atm)⁻¹] | | | | | |
| Exposure | Incident angle θ₁ of first light flux [deg] | | 6.5 | | | |
| condition | Incident angle θ₂ of second light flux [deg] | | 9.5 | | | |
| Evaluation result | Hologram | Diffraction | 32 | 32 | 35 | 35 |
| | characteristic | efficiency [%] | | | | |
| | Storage stability | Retention [%] | 47 | 86 | 56 | 69 |
| | | | Example | Example | Example | Example |
| | | | 1-10 | 1-11 | 1-12 | 1-13 |
| Photosensitive | Monomer | EA-0200 | 35.19 | | 34.04 | |
| composition | | EACz | 7.84 | | 7.58 | |
| | Binder resin | SN-55T | 18.53 | | 17.92 | |
| | Plasticizer | SDE | 28.68 | | 27.75 | |
| | Sensitizing dye | MB | 0.45 | | 0.43 | |
| | Initiator | I0591 | — | | — | |
| | | (onium salt- | | | | |
| | | based | | | | |
| | | initiator) | | | | |
| | | Irgacure 290 | — | | — | |
| | | (onium salt- | | | | |
| | | based | | | | |
| | | initiator) | | | | |
| | | T2041 | — | | — | |
| | | (onium salt- | | | | |
| | | based | | | | |
| | | initiator) | | | | |
| | | CPI-100P | 4.10 | | — | |
| | | (onium salt- | | | | |
| | | based | | | | |
| | | initiator) | | | | |
| | | CPI-310B | — | | 7.23 | |
| | | (onium salt- | | | | |
| | | based | | | | |
| | | initiator) | | | | |
| | | P3B | 3.27 | | 3.16 | |
| | | (organic boron | | | | |
| | | salt-based | | | | |
| | | initiator) | | | | |
| | Chain transfer | 2-MBO | 0.54 | | 0.52 | |
| | agent | | | | | |
| | Polymerization | PT | 0.08 | | 0.07 | |
| | inhibitor | | | | | |
| | UV sensitizer | UVS-1331 | 1.33 | | 1.29 | |
| | Solid concentration | | 27.6 | | | |
| Base material (1) | Material | | COP | COP | COP | COP |
| | Thickness [μm] | | 50 | 50 | 50 | 50 |
| | Oxygen permeability | | 590 | 590 | 590 | 590 |
| | [cm3 · (m2 · day · atm)⁻¹] | | | | | |
| Base material (2) | Material | | Glass | PET | Glass | PET |
| | Thickness [μm] | | 1000 | 100 | 1000 | 100 |
| | Oxygen permeability | | — | 15 | — | 15 |
| | [cm³ · (m² · day · atm)⁻¹] | | | | | |
| Exposure | Incident angle θ₁ of first light flux [deg] | | 6.5 | | | |
| condition | Incident angle θ₂ of second light flux [deg] | | 9.5 | | | |
| Evaluation result | Hologram | Diffraction | 39 | 39 | 36 | 36 |
| | characteristic | efficiency [%] | | | | |
| | Storage stability | Retention [%] | 60 | 80 | 43 | 58 |

TABLE 3

| | | | Example | Example | Example | Example | Example |
|---|---|---|---|---|---|---|---|
| | | | 2-1 | 2-2 | 3-1 | 3-2 | 4-1 |
| Photosensitive | Monomer | Compound | 23.00 | 23.00 | 22.77 | 22.77 | 7.48 |
| composition | | (6-3) | | | | | |
| | | Compound | 0 | 0 | 19.18 | 19.18 | 0 |
| | | (11-10) | | | | | |
| | | EA-0200 | 18.39 | 18.39 | 0 | 0 | 33.59 |
| | | EACz | 0 | 0 | 0 | 0 | 0 |
| | Binder resin | SN-55T | 17.82 | 17.82 | 17.65 | 17.65 | 17.68 |
| | Plasticizer | EX212L | 27.59 | 27.59 | 27.33 | 27.33 | 27.38 |
| | | SDE | 0 | 0 | 0 | 0 | 0 |
| | Sensitizing | MB | 0.43 | 0.43 | 0.43 | 0.43 | 0.43 |
| | dye | RB | 0 | 0 | 0 | 0 | 0 |
| | | SFO | 0 | 0 | 0 | 0 | 0.07 |
| | | AOG | 0 | 0 | 0 | 0 | 0.71 |
| | | BCp-1 | 0 | 0 | 0 | 0 | 0 |
| | Initiator | I0591 | 7.75 | 7.75 | 7.68 | 7.68 | 7.69 |
| | | (onium | | | | | |
| | | salt-based | | | | | |
| | | initiator) | | | | | |
| | | Irgacure | 0 | 0 | 0 | 0 | 0 |
| | | 290 | | | | | |
| | | (onium | | | | | |
| | | salt-based | | | | | |
| | | initiator) | | | | | |
| | | P3B | 3.15 | 3.15 | 3.11 | 3.11 | 3.12 |
| | | (organic | | | | | |
| | | boron salt- | | | | | |
| | | based | | | | | |
| | | initiator) | | | | | |
| | Chain transfer | 2-MBO | 0.52 | 0.52 | 0.51 | 0.51 | 0.51 |
| | agent | | | | | | |
| | Polymerization | PT | 0.07 | 0.07 | 0.07 | 0.07 | 0.07 |
| | inhibitor | | | | | | |
| | UV sensitizer | UVS-1331 | 1.28 | 1.28 | 1.27 | 1.27 | 1.27 |
| | Color Former | PG | 0 | 0 | 0 | 0 | 0 |
| | Solid concentration | | 27.6 | 27.6 | 27.6 | 27.6 | 27.6 |
| Base material | Material | | PET | COP | PET | COP | PET |
| (1) | Thickness [μm] | | 100 | 50 | 100 | 50 | 100 |
| | Oxygen permeability | | 15 | 590 | 15 | 590 | 15 |
| | [cm³ · (m² · day · atm)⁻¹] | | | | | | |
| Base material | Material | | Glass | Glass | Glass | Glass | Glass |
| (2) | Thickness [μm] | | 1000 | 1000 | 1000 | 1000 | 1000 |
| | Oxygen permeability | | — | — | — | — | — |
| | [cm³ · (m² · day · atm)⁻¹] | | | | | | |
| Exposure | Incident angle θ₁ of first | | 6.5 | | | | |
| condition | light flux [deg] | | | | | | |
| | Incident angle θ₂ of | | 9.5 | | | | |
| | second light flux [deg] | | | | | | |
| Evaluation | Hologram | Diffraction | 39 | 37 | 26 | 29 | 42 |
| result | characteristic | efficiency | | | | | |
| | | [%] | | | | | |
| | Storage | Retention | 80 | 100 | 38 | 78 | 44 |
| | stability | [%] | | | | | |

| | | | Example | Comparative | Comparative | Comparative |
|---|---|---|---|---|---|---|
| | | | 4-2 | Example 2 | Example 3 | Example 4 |
| Photosensitive | Monomer | Compound | 7.48 | 23.00 | 22.77 | 7.48 |
| composition | | (6-3) | | | | |
| | | Compound | 0 | 0 | 19.18 | 0 |
| | | (11-10) | | | | |
| | | EA-0200 | 33.59 | 18.39 | 0 | 33.59 |
| | | EACz | 0 | 0 | 0 | 0 |
| | Binder resin | SN-55T | 17.68 | 17.82 | 17.65 | 17.68 |
| | Plasticizer | EX212L | 27.38 | 27.59 | 27.33 | 27.38 |
| | | SDE | 0 | 0 | 0 | 0 |
| | Sensitizing | MB | 0.43 | 0.43 | 0.43 | 0.43 |
| | dye | RB | 0 | 0 | 0 | 0 |
| | | SFO | 0.07 | 0 | 0 | 0.07 |
| | | AOG | 0.71 | 0 | 0 | 0.71 |
| | | BCp-1 | 0 | 0 | 0 | 0 |
| | Initiator | I0591 | 7.69 | 7.75 | 7.68 | 7.69 |
| | | (onium | | | | |
| | | salt-based | | | | |
| | | initiator) | | | | |
| | | Irgacure | 0 | 0 | 0 | 0 |
| | | 290 | | | | |
| | | (onium | | | | |
| | | salt-based | | | | |
| | | initiator) | | | | |
| | | P3B | 3.12 | 3.15 | 3.11 | 3.12 |
| | | (organic | | | | |
| | | boron salt- | | | | |
| | | based | | | | |
| | | initiator) | | | | |
| | Chain transfer | 2-MBO | 0.51 | 0.52 | 0.51 | 0.51 |
| | agent | | | | | |
| | Polymerization | PT | 0.07 | 0.07 | 0.07 | 0.07 |
| | inhibitor | | | | | |
| | UV sensitizer | UVS-1331 | 1.27 | 1.28 | 1.27 | 1.27 |
| | Color Former | PG | 0 | 0 | 0 | 0 |
| | Solid concentration | | 27.6 | 27.6 | 27.6 | 27.6 |
| Base material | Material | | COP | PVA/COP | PVA/COP | PVA/COP |
| (1) | Thickness [μm] | | 50 | 52.5 | 52.5 | 52.5 |
| | Oxygen permeability | | 590 | 0.1 | 0.1 | 0.1 |
| | [cm³ · (m² · day · atm)⁻¹] | | | | | |
| Base material | Material | | Glass | Glass | Glass | Glass |
| (2) | Thickness [μm] | | 1000 | 1000 | 1000 | 1000 |
| | Oxygen permeability | | — | — | — | — |
| | [cm³ · (m² · day · atm)⁻¹] | | | | | |
| Exposure | Incident angle θ₁ of first | | 6.5 | | | |
| condition | light flux [deg] | | | | | |
| | Incident angle θ₂ of | | 9.5 | | | |
| | second light flux [deg] | | | | | |
| Evaluation | Hologram | Diffraction | 59 | 24 | 13 | 41 |
| result | characteristic | efficiency | | | | |
| | | [%] | | | | |
| | Storage | Retention | 50 | 14 | 17 | 0 |
| | stability | [%] | | | | |

TABLE 4

| | | | Example | Example | Example | Example | Example |
|---|---|---|---|---|---|---|---|
| | | | 5-1 | 5-2 | 5-3 | 5-4 | 5-5 |
| Photosensitive | Monomer | Compound | 0 | 0 | 0 | 0 | 0 |
| composition | | (6-3) | | | | | |
| | | Compound | 0 | 0 | 0 | 0 | 0 |
| | | (11-10) | | | | | |
| | | EA-0200 | 31.69 | 31.69 | 31.69 | 31.69 | 31.69 |
| | | EACz | 7.06 | 7.06 | 7.06 | 7.06 | 7.06 |
| | Binder resin | SN-55T | 16.69 | 16.69 | 16.69 | 16.69 | 16.69 |
| | Plasticizer | EX212L | 12.92 | 12.92 | 12.92 | 12.92 | 12.92 |
| | | SDE | 12.92 | 12.92 | 12.92 | 12.92 | 12.92 |
| | Sensitizing dye | MB | 0.47 | 0.47 | 0.47 | 0.47 | 0.47 |
| | | RB | 0 | 0 | 0 | 0 | 0 |
| | | SFO | 0 | 0 | 0 | 0 | 0 |
| | | AOG | 0 | 0 | 0 | 0 | 0 |
| | | BCp-1 | 0.15 | 0.15 | 0.15 | 0.15 | 0.15 |
| | Initiator | I0591 (onium | 0 | 0 | 0 | 0 | 0 |
| | | salt-based | | | | | |
| | | initiator) | | | | | |
| | | Irgacure 290 | 9.95 | 9.95 | 9.95 | 9.95 | 9.95 |
| | | (onium salt- | | | | | |
| | | based | | | | | |
| | | initiator) | | | | | |
| | | P3B (organic | 2.95 | 2.95 | 2.95 | 2.95 | 2.95 |
| | | boron salt- | | | | | |
| | | based | | | | | |
| | | initiator) | | | | | |
| | Chain transfer | 2-MBO | 0.48 | 0.48 | 0.48 | 0.48 | 0.48 |
| | agent | | | | | | |
| | Polymerization | PT | 0.07 | 0.07 | 0.07 | 0.07 | 0.07 |
| | inhibitor | | | | | | |
| | UV sensitizer | UVS-1331 | 1.20 | 1.20 | 1.20 | 1.20 | 1.20 |
| | Color Former | PG | 3.45 | 3.45 | 3.45 | 3.45 | 3.45 |
| | Solid concentration | | 27.6 | 27.6 | 27.6 | 27.6 | 27.6 |
| Base material | Material | | HC-PC | COP | HC-PC | COP | PVA/COP |
| (1) | Thickness [μm] | | 110 | 50 | 110 | 50 | 52.5 |
| | Oxygen permeability | | 390 | 590 | 390 | 590 | 0.1 |
| | [cm³ · (m² · day · atm)⁻¹ | | | | | | |
| Base material | Material | | Glass | Glass | PET | PET | PET |
| (2) | Thickness [μm] | | 1000 | 1000 | 100 | 100 | 100 |
| | Oxygen permeability | | — | — | 15 | 15 | 15 |
| | [cm³ · (m² · day · atm)⁻¹] | | | | | | |
| Exposure | Incident angle θ₁ of first | | 6.5 | | | | |
| condition | light flux [deg] | | | | | | |
| | Incident angle θ₂ of second | | 9.5 | | | | |
| | light flux [deg] | | | | | | |
| Evaluation | Hologram | Diffraction | 36 | 60 | 36 | 60 | 56 |
| result | characteristic | efficiency | | | | | |
| | | [%] | | | | | |
| | Storage stability | Retention | 98 | 98 | 88 | 84 | 103 |
| | | [%] | | | | | |

| | | | | | | Comparative |
|---|---|---|---|---|---|---|
| | | | Example | Example | Example | Example |
| | | | 5-6 | 5-7 | 5-8 | 5 |
| Photosensitive | Monomer | Compound | 0 | 0 | 0 | 0 |
| composition | | (6-3) | | | | |
| | | Compound | 0 | 0 | 0 | 0 |
| | | (11-10) | | | | |
| | | EA-0200 | 32.57 | 32.57 | 32.57 | 32.57 |
| | | EACz | 7.26 | 7.26 | 7.26 | 7.26 |
| | Binder resin | SN-55T | 17.15 | 17.15 | 17.15 | 17.15 |
| | Plasticizer | EX212L | 13.28 | 13.28 | 13.28 | 13.28 |
| | | SDE | 13.28 | 13.28 | 13.28 | 13.28 |
| | Sensitizing dye | MB | 0.49 | 0.49 | 0.49 | 0.49 |
| | | RB | 0 | 0 | 0 | 0 |
| | | SFO | 0 | 0 | 0 | 0 |
| | | AOG | 0 | 0 | 0 | 0 |
| | | BCp-1 | 0.16 | 0.16 | 0.16 | 0.16 |
| | Initiator | I0591 (onium | 7.46 | 7.46 | 7.46 | 7.46 |
| | | salt-based | | | | |
| | | initiator) | | | | |
| | | Irgacure 290 | 0 | 0 | 0 | 0 |
| | | (onium salt- | | | | |
| | | based | | | | |
| | | initiator) | | | | |
| | | P3B (organic | 3.03 | 3.03 | 3.03 | 3.03 |
| | | boron salt- | | | | |
| | | based | | | | |
| | | initiator) | | | | |
| | Chain transfer | 2-MBO | 0.50 | 0.50 | 0.50 | 0.50 |
| | agent | | | | | |
| | Polymerization | PT | 0.07 | 0.07 | 0.07 | 0.07 |
| | inhibitor | | | | | |
| | UV sensitizer | UVS-1331 | 1.23 | 1.23 | 1.23 | 1.23 |
| | Color Former | PG | 3.54 | 3.54 | 3.54 | 3.54 |
| | Solid concentration | | 27.6 | 27.6 | 27.6 | 27.6 |
| Base material | Material | | COP | COP | PVA/COP | PVA/COP |
| (1) | Thickness [μm] | | 50 | 50 | 52.5 | 52.5 |
| | Oxygen permeability | | 590 | 590 | 0.1 | 0.1 |
| | [cm³ · (m² · day · atm)⁻¹ | | | | | |
| Base material | Material | | Glass | PET | PET | Glass |
| (2) | Thickness [μm] | | 1000 | 100 | 100 | 1000 |
| | Oxygen permeability | | — | 15 | 15 | — |
| | [cm³ · (m² · day · atm)⁻¹] | | | | | |
| Exposure | Incident angle θ₁ of first | | 6.5 | | | |
| condition | light flux [deg] | | | | | |
| | Incident angle θ₂ of second | | 9.5 | | | |
| | light flux [deg] | | | | | |
| Evaluation | Hologram | Diffraction | 48 | 48 | 48 | 48 |
| result | characteristic | efficiency | | | | |
| | | [%] | | | | |
| | Storage stability | Retention | 95 | 100 | 95 | 0 |
| | | [%] | | | | |

In Tables 1, 2, 3, and 4, the numerical value of each component in the photosensitive composition indicates the amount by “mass %” with respect to 100 mass % of the total of the components excluding the methyl ethyl ketone/ethanol mixed solvent used in the solvent of the coating liquid, and the solid concentration indicates the amount by “mass %” of the total of the components excluding the methyl ethyl ketone/ethanol mixed solvent with respect to 100 mass % of the total of the components in a table contained in the methyl ethyl ketone/ethanol mixed solvent and the coating liquid.

The incident angle θ₁ of the first light flux and the incident angle θ₂ of the second light flux are defined as described in the second embodiment (see FIG. 4).

Details of each material described in Tables 1, 2, 3, and 4 are as follows.

<Radical Polymerizable Monomer>

EA-0200: bisphenoxyethanol fluorenediacrylate (manufactured by Osaka Gas Chemicals Co., Ltd., product name: EA-0200) (polymerizable compound represented by chemical formula (10-1) in first embodiment)

EACz: 2-(9H-carbazol-9-yl)ethyl acrylate (manufactured by SIGMA-ALDRICH Co. LLC) (polymerizable compound represented by chemical formula (0-4) in first embodiment)

Compound (6-3): Polymerizable compound represented by chemical formula (6-3) in first embodiment

Compound (11-10): Polymerizable compound represented by chemical formula (11-10) in first embodiment

<Binder Resin>

SN-55T: polyvinyl acetate (manufactured by Denka Company Limited, product name: DENKA SAKNOHOL SN-55T (average polymerization degree: 5500))

<Plasticizer>

SDE: diethyl sebacate (manufactured by FUJIFILM Wako Pure Chemical Corporation)

EX 212L: 1,6-hexanediol diglycidyl ether (manufactured by Nagase ChemteX Corporation, product name: EX 212L)

<Sensitizing Dye>

MB: methylene blue (manufactured by SIGMA-ALDRICH Co. LLC)

RB: rose bengal (manufactured by SIGMA-ALDRICH Co. LLC)

SFO: Safranin O (manufactured by SIGMA-ALDRICH Co. LLC)

AOG: Astrazon Orange G (manufactured by SIGMA-ALDRICH Co. LLC)

BCp-1: (2E,5E)-2,5-bis[(4-(diethylamino)phenyl)methylene]cyclopentanone (manufactured by Acros Organics)

<Initiator>

I0591: 4-isopropyl-4′-methyldiphenyliodonium tetrakis(pentafluorophenyl)borate (onium salt-based initiator) (manufactured by Tokyo Chemical Industry Co., Ltd.)

Irgacure 290: triarylsulfonium borate salt (onium salt-based initiator) (manufactured by BASF Japan)

T2041: tri-p-tolylsulfonium hexafluorophosphate (onium salt-based initiator) (manufactured by Tokyo Chemical Industry Co., Ltd., product code: T2041)

CPI-100 P: diphenyl 4-thiophenoxyphenylsulfonium hexafluorophosphate (onium salt-based initiator) (manufactured by San-Apro Ltd., product name: CPI-100P)

CPI-310B: triarylsulfonium borate salt (onium salt-based initiator) (manufactured by San-Apro Ltd., product name: CPI-310B)

P3B: tetrabutylammonium butyltriphenylborate (organic boron salt-based initiator) (manufactured by Showa Denko K.K., product name: P3B)

<Chain Transfer Agent>

2-MBO: 2-mercaptobenzoxazole (manufactured by Tokyo Chemical Industry Co., Ltd.)

<Polymerization Inhibitor>

PT: phenothiazine (manufactured by FUJIFILM Wako Pure Chemical Corporation)

<UV Sensitizer>

UVS-1331: 9,10 dibutoxyanthracene (manufactured by Kawasaki Kasei Chemicals Ltd., product name: UVS-1331)

<Color Former>

PG: polyethylene glycol

<Base Material>

PET: polyethylene terephthalate (manufactured by Panac Co., Ltd., product name: NP100A)

HC-PC: polycarbonate with hard coat (manufactured by TEIJIN LIMITED, product name: XLC806C)

COP: cycloolefin polymer (manufactured by Zeon Corporation, product name/film thickness: ZF14-50/50 μm, ZF14-23/23 μm, ZF14-13/film thickness 13 μm)

PVA/COP: An aqueous solution in which PVA (manufactured by The Nippon Synthetic Chemical Industry Co., Ltd., product name: NH-18, polyvinyl alcohol) was dissolved was applied onto a COP film of ZF14-50 and dried to form a film having a thickness of 2.5 μm.

The following is found from Tables 1, 2, 3, and 4.

If the oxygen permeability of the film (base material) measured in an environment at 23° C. and 0% RH is more than 0.1 cm³·(m²·day·atm)⁻¹, deterioration of the storage stability in an unexposed state can be suppressed.

If the oxygen permeability of at least one of the film (base material (1)) provided on the first surface of the photosensitive layer and the film (base material (2)) provided on the second surface of the photosensitive layer is more than 0.1 cm³·(m²·day·atm)⁻¹, deterioration of the storage stability in an unexposed state can be suppressed.

If the photosensitive layer 12 contains a compound represented by the general formula (1), the diffraction efficiency can be improved.

### Example 6-1

The radical polymerizable monomers, the binder resin, the plasticizer, the sensitizing dye, the onium salt-based initiator, the organic boron salt-based initiator, the chain transfer agent, the polymerization inhibitor, and the UV sensitizer described in Table 5 were prepared, and these were weighed out at a proportion described in Table 5. Furthermore, an HC-PC film having a thickness of 110 μm was used in place of the PET film having a thickness of 100 μm. A hologram recording medium was produced in a manner similar to that in Example 1-1 except for these procedures.

### Example 6-2

A hologram recording medium was produced in a manner similar to that in Example 1-1 except that a COP film having a thickness of 23 μm was used in place of the PET film having a thickness of 100 μm.

### Example 6-3

The photosensitive composition was applied, with a bar coater so as to have a dry film thickness of 5 μm, onto a PVA film of a PVA/COP film obtained by stacking a PVA film having a thickness of 2.5 μm and a COP film having a thickness of 50 μm, then the thin film surface of the photosensitive layer including the photosensitive composition was pressure-bonded onto a glass substrate having a thickness of 1.0 mm, and only the upper COP film was peeled off from the pressure-bonded body to obtain a hologram recording medium in which the PVA film, the photosensitive layer, and the glass were stacked.

### Example 6-4

A photosensitive composition was prepared in a manner similar to that in Example 6-1 except that the addition of the onium salt-based initiator was omitted. Furthermore, a hologram recording medium was produced in a manner similar to that in Example 1-1 except that an HC-PC film having a thickness of 110 μm was used in place of the PET film having a thickness of 100 μm.

[Evaluation of Characteristics of Hologram]

First, the hologram recording medium immediately after the production was subjected to two-beam interference exposure using a diode-pumped solid-state laser having exposure wavelengths of 457 nm, 532 nm, and 660 nmnm. Thereafter, the entire surface was irradiated with UV light to cure the uncured monomer, and the refractive index distribution was fixed in the hologram recording medium. As a result, a hologram optical element was obtained. For the exposure, an optical system (optical system for hologram recording) illustrated in FIG. 3 was used. Subsequently, the refractive index modulation amount (Δn) of the produced hologram recording medium was determined with the following evaluation method using the theoretical formula by Kogelnik. Table 5 shows the results.

(Evaluation of Refractive Index Modulation (an))

On the basis of the theoretical formula by Kogelnik described in Bell Syst. Tech. J., 48, 2909 (1969), Δn was determined from the maximum diffraction efficiency, the wavelength of the reproduction illumination light, the incident angle of the reproduction illumination light, the grating period of the diffraction grating, the grating inclination angle of the diffraction grating, the film thickness of the hologram layer of the hologram optical element, and the average refractive index of the hologram layer of the hologram optical element, which were known, using the relational expression for the time of satisfying the Bragg condition in a reflection-type volume phase hologram among the Δn, the maximum diffraction efficiency, the wavelength of reproduction illumination light, the incident angle of the reproduction illumination light, the film thickness of the hologram layer of the hologram optical element, and the average refractive index of the hologram layer of the hologram optical element.

Furthermore, the following devices were used as the diode-pumped solid-state laser and the UV light irradiation device, and for measurement of Δn.

<Diode-Pumped Solid-State Laser>

660 nm Diode-pumped solid-state laser: manufactured by Cobolt AB, product name: Flamenco 660 nm

532 nm Diode-pumped solid-state laser: manufactured by Cobolt AB, product name: Samba 532 nm

457 nm Diode-pumped solid-state laser: manufactured by Melles Griot, product name: 85 BLS 601 457 nm

<UV Light Irradiation Device>

CV-1LC-G: manufactured by Heraeus, product name: CV-1LC-G, LED lamp used: Semray UV4003, integrated UV light amount: 4 J·cm⁻², wavelength: 365 nm)

<Δn Measuring Device>

Light source: manufactured by MATSU PHOTONICS K.K., product name: LIGHTNINGCURE, spot light source LC8

Spectrometer: manufactured by Ocean Optics, Inc., product name: USB4000 Miniature Fiber Optic Spectrometer

[Evaluation of Maximum Difference in Peak Wavelength]

The diffraction peak wavelength of the hologram formed in the hologram recording area (area: 180 mm²) was measured, and the difference between the maximum diffraction wavelength and the minimum diffraction wavelength obtained was regarded as the maximum difference in peak wavelength. Table 5 shows the results.

TABLE 5

| | | | Example | Example | Example | Example |
|---|---|---|---|---|---|---|
| | | | 6-1 | 6-2 | 6-3 | 6-4 |
| Photosensitive | Monomer | EA-0200 | 36.80 | 36.80 | 36.80 | 36.80 |
| composition | | EACz | 8.20 | 8.20 | 8.20 | 8.20 |
| | Binder resin | SN-55T | 19.38 | 19.38 | 19.38 | 19.38 |
| | Plasticizer | EX212L | 30.00 | 30.00 | 30.00 | 30.00 |
| | Sensitizing dye | RB | 2.25 | 2.25 | 2.25 | 2.25 |
| | Initiator | I0591 (onium salt-based | 8.43 | 8.43 | 8.43 | 0 |
| | | initiator) | | | | |
| | | P3B (organic boron salt- | 3.42 | 3.42 | 3.42 | 3.42 |
| | | based initiator) | | | | |
| | Chain transfer agent | 2-MBO | 0.56 | 0.56 | 0.56 | 0.56 |
| | Polymerization inhibitor | PT | 0.15 | 0.15 | 0.15 | 0.15 |
| | UV sensitizer | UVS-1331 | 2.67 | 2.67 | 2.67 | 2.67 |

| | Solid concentration | 30.0 | 30.0 | 30.0 | 30.0 |
|---|---|---|---|---|---|
| Base material | Material | HC-PC | COP | PVA | HC-PC |
| | Thickness [μm] | 110 | 23 | 2.5 | 110 |
| | Oxygen permeability [cm³ · (m² · day · atm)⁻¹] | 390 | 1300 | Not | 390 |
| | | | | measurable | |
| Exposure condition | Exposure wavelength [nm] | 532 | 532 | 532 | 532 |
| Exposure condition | Incident angle θ₁ of first light flux [deg] | 6.5 | | | |
| | Incident angle θ₂ of second light flux [deg] | 9.5 | | | |
| Evaluation result | Δn | 0.032 | 0.028 | 0.029 | Diffracted light |
| | | | | | was not detected |
| | Maximum difference in peak | 18 | 8 | 13 | Diffracted light |
| | wavelength [nm] | | | | was not detected |

In Table 5, the numerical value of each component in the photosensitive composition indicates the amount by “parts by mass” of the component excluding the methyl ethyl ketone/ethanol mixed solvent used in the solvent of the coating liquid, and the solid concentration indicates the amount by “mass %” of the total of the components excluding the methyl ethyl ketone/ethanol mixed solvent with respect to 100 mass % of the total of the components in the table contained in the methyl ethyl ketone/ethanol mixed solvent and the coating liquid.

The incident angle θ₁ of the first light flux and the incident angle θ₂ of the second light flux are defined as described in the second embodiment (see FIG. 4).

The details of each material described in Table 5 are similar to those of each material described in Tables 1, 2, 3, and 4.

The following is found from Table 5.

If both the organic boron salt-based initiator and the onium salt-based initiator are contained in the photosensitive composition, the refractive index modulation amount (Δn) can be increased.

Furthermore, the maximum difference in peak wavelength can be reduced. Therefore, the uniformity of a diffraction peak wavelength in the interference exposure area can be enhanced.

Although the embodiments of the present disclosure are heretofore described specifically, the present disclosure is not limited to the above-described embodiments, and various modifications based on the technical idea of the present disclosure can be made.

For example, the configurations, the methods, the steps, the shapes, the materials, the numerical values, and the like described in the above-described embodiments are merely examples, and a configuration, a method, a step, a shape, a material, a numerical value, and the like that are different from those in the above-described embodiments may also be used as necessary.

Furthermore, the configurations, the methods, the steps, the shapes, the materials, the numerical values, and the like of the above-described embodiments can be combined with each other without departing from the gist of the present disclosure.

In the numerical value ranges described in stages in the above-described embodiments, an upper limit or a lower limit of a numerical value range of a certain stage may be replaced with an upper limit or a lower limit of a numerical value range of another stage. The materials exemplified in the above-described embodiments can be used alone or in combination of two or more kinds thereof unless otherwise specified.

Furthermore, the present disclosure can also employ the following configurations.

(1)

A hologram recording medium including
a base material and a photosensitive layer,
the base material having an oxygen permeability of more than 0.1 cm³·(m²·day·atm)⁻¹ and 10000 cm³·(m²·day·atm)⁻¹ or less, the oxygen permeability measured in an environment at 23° C. and 0% RH,
the photosensitive layer containing a polymerizable compound, the polymerizable compound containing a compound represented by a general formula (1) described below.

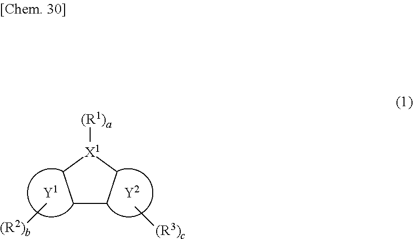

In the general formula (1), X¹ represents an oxygen atom, a nitrogen atom, a phosphorus atom, a carbon atom, or a silicon atom. In a case where X¹ represents an oxygen atom, a is 0, in a case where X¹ represents a nitrogen atom or a phosphorus atom, a is 1, and in a case where X¹ represents a carbon atom or a silicon atom, a is 2.

Y¹ and Y² each represent a benzene ring or a naphthalene ring. In a case where Y¹ and/or Y² represents a benzene ring, b or c corresponding to Y¹ and/or Y² representing the benzene ring is 4. In a case where Y¹ and/or Y² represents a naphthalene ring, b and/or c corresponding to Y¹ and/or Y² representing the naphthalene ring is 6.

R¹ to R³ each represent hydrogen or a substituent represented by *—Z¹(R⁴)_{d} (in which * represents a binding site). In a case where a plurality of R¹s, a plurality of R²s, and a plurality of R³s are present, the plurality of R¹s, the plurality of R²s, and the plurality of R³s may be of an identical kind or of different kinds, and all of the plurality of R¹s, the plurality of R²s, and the plurality of R³s in the general formula (1) are not simultaneously hydrogen.

Z¹ represents a single bond, a divalent or higher saturated hydrocarbon group, or a divalent or higher unsaturated hydrocarbon group, and the divalent or higher saturated hydrocarbon group or the divalent or higher unsaturated hydrocarbon group may contain an ether bond and/or a thioether bond. In a case where Z¹ represents a single bond, d is 1, and in a case where Z¹ represents a divalent or higher saturated hydrocarbon group or a divalent or higher unsaturated hydrocarbon group, d is an integer of 1 or more.

R⁴ represents hydrogen or a polymerizable substituent. In a case where a plurality of R⁴s is present, the plurality of R⁴s may be of an identical kind or of different kinds, and all of the plurality of R⁴s in the general formula (1) is not simultaneously hydrogen.

(2)

The hologram recording medium according to (1), in which

the photosensitive layer further contains a photopolymerizable initiator, and

the photopolymerizable initiator contains at least one of an organic boron salt-based initiator or an onium salt-based initiator.

(3)

The hologram recording medium according to (1), in which

the photosensitive layer further contains a photopolymerizable initiator, and

the photopolymerization initiator contains an organic boron salt-based initiator and an onium salt-based initiator.

(4)

The hologram recording medium according to any one of (1) to (3), in which the compound is represented by a general formula (1-1) described below.

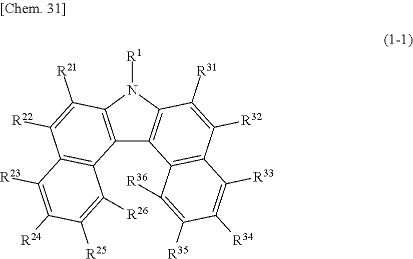

In the general formula (1-1), R¹, R²¹ to R²⁶, and R³¹ to R³⁶ represent hydrogen or a substituent represented by *—Z¹(R⁴)_{d} (in which * represents a binding site). R¹, R²¹ to R²⁶, and R³¹ to R³⁶ may be of an identical kind or of different kinds. However, R¹, R²¹ to R²⁶, and R³¹ to R³⁶ in the general formula (1-1) are not simultaneously hydrogen.

Z¹ represents a single bond, a divalent or higher saturated hydrocarbon group, or a divalent or higher unsaturated hydrocarbon group, and the divalent or higher saturated hydrocarbon group or the divalent or higher unsaturated hydrocarbon group may contain an ether bond and/or a thioether bond. In a case where Z¹ represents a single bond, d is 1, and in a case where Z¹ represents a divalent or higher saturated hydrocarbon group or a divalent or higher unsaturated hydrocarbon group, d is an integer of 1 or more.

R⁴ represents hydrogen or a polymerizable substituent. In a case where a plurality of R⁴s is present, the plurality of R⁴s may be of an identical kind or of different kinds, and all of the plurality of R⁴s in the general formula (1-1) is not simultaneously hydrogen.

(5)

The hologram recording medium according to any one of (1) to (3), in which the compound is represented by a general formula (1-2) described below.

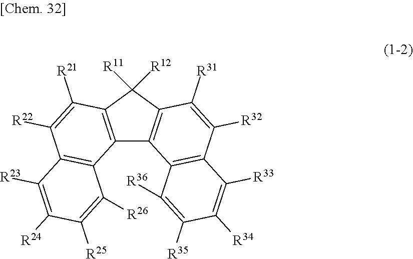

In the general formula (1-2), R¹¹, R¹², R²¹ to R²⁶, and R³¹ to R³⁶ represent hydrogen or a substituent represented by *—Z¹(R⁴)_{d} (in which * represents a binding site). R¹¹, R¹², R²¹ to R²⁶, and R³¹ to R³⁶ may be of an identical kind or of different kinds. However, R¹¹, R¹², R²¹ to R²⁶, and R³¹ to R³⁶ in the general formula (1-2) are not simultaneously hydrogen.

Z¹ represents a single bond, a divalent or higher saturated hydrocarbon group, or a divalent or higher unsaturated hydrocarbon group, and the divalent or higher saturated hydrocarbon group or the divalent or higher unsaturated hydrocarbon group may contain an ether bond and/or a thioether bond. In a case where Z¹ represents a single bond, d is 1, and in a case where Z¹ represents a divalent or higher saturated hydrocarbon group or a divalent or higher unsaturated hydrocarbon group, d is an integer of 1 or more.

R⁴ represents hydrogen or a polymerizable substituent. In a case where a plurality of R⁴s is present, the plurality of R⁴s may be of an identical kind or of different kinds, and all of the plurality of R⁴s in the general formula (1-2) is not simultaneously hydrogen.

(6)

The hologram recording medium according to any one of (1) to (5), in which the photosensitive layer further contains a binder resin.

(7)

The hologram recording medium according to any one of (1) to (3), in which Y¹ and Y² in the general formula (1) are not simultaneously a benzene ring.

(8)

The hologram recording medium according to claim 1, in which the oxygen permeability of the base material measured in the environment at 23° C. and 0% RH is more than 5 cm³·(m²·day·atm)⁻¹ and 10000 cm³·(m²·day·atm)⁻¹ or less.

(9)

A hologram optical element including the hologram recording medium according to any one of (1) to (8), in which

the polymerizable compound is polymerized.

(10)

An optical device including the hologram optical element according to (9).

(11)

An optical component including the hologram optical element according to (9).

(12)

A method for forming a hologram diffraction grating, the method including
selectively reacting a hologram recording medium including a base material and a photosensitive layer, by using an electromagnetic ray having an amplitude modulated spatially,
the base material having an oxygen permeability of more than 0.1 cm³·(m²·day·atm)⁻¹ and 10000 cm³·(m²·day·atm)⁻¹ or less, the oxygen permeability measured in an environment at 23° C. and 0% RH,
the photosensitive layer containing a polymerizable compound, the polymerizable compound represented by a general formula (1) described below.

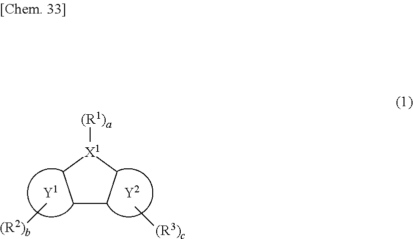

In the general formula (1), X¹ represents an oxygen atom, a nitrogen atom, a phosphorus atom, a carbon atom, or a silicon atom. In a case where X¹ represents an oxygen atom, a is 0, in a case where X¹ represents a nitrogen atom or a phosphorus atom, a is 1, and in a case where X¹ represents a carbon atom or a silicon atom, a is 2.

Y¹ and Y² each represent a benzene ring or a naphthalene ring. In a case where Y¹ and/or Y² represents a benzene ring, b or c corresponding to Y¹ and/or Y² representing the benzene ring is 4. In a case where Y¹ and/or Y² represents a naphthalene ring, b and/or c corresponding to Y¹ and/or Y² representing the naphthalene ring is 6.

R¹ to R³ each represent hydrogen or a substituent represented by *—Z¹(R⁴)_{d} (in which * represents a binding site). In a case where a plurality of R¹s, a plurality of R²s, and a plurality of R³s are present, the plurality of R¹s, the plurality of R²s, and the plurality of R³s may be of an identical kind or of different kinds, and all of the plurality of R¹s, the plurality of R²s, and the plurality of R³s in the general formula (1) are not simultaneously hydrogen.

Z¹ represents a single bond, a divalent or higher saturated hydrocarbon group, or a divalent or higher unsaturated hydrocarbon group, and the divalent or higher saturated hydrocarbon group or the divalent or higher unsaturated hydrocarbon group may contain an ether bond and/or a thioether bond. In a case where Z¹ represents a single bond, d is 1, and in a case where Z¹ represents a divalent or higher saturated hydrocarbon group or a divalent or higher unsaturated hydrocarbon group, d is an integer of 1 or more.

R⁴ represents hydrogen or a polymerizable substituent. In a case where a plurality of R⁴s is present, the plurality of R⁴s may be of an identical kind or of different kinds, and all of the plurality of R⁴s in the general formula (1) is not simultaneously hydrogen.

### REFERENCE SIGNS LIST

10 Hologram recording medium
11 Base material
12 Photosensitive layer
13 Release layer
14 Glass base material

1. A hologram recording medium comprising:
a base material; and a photosensitive layer,
the base material having an oxygen permeability of more than 0.1 cm³·(m²·day·atm)⁻¹ and 10000 cm³·(m²·day·atm)⁻¹ or less, the oxygen permeability measured in an environment at 23° C. and 0% RH,
the photosensitive layer containing a polymerizable compound, the polymerizable compound containing a compound represented by a general formula (1) described below: 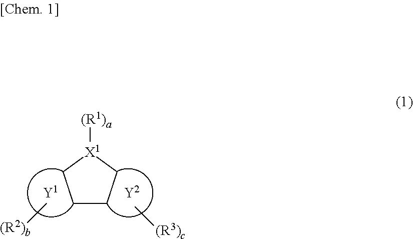
wherein X¹ represents an oxygen atom, a nitrogen atom, a phosphorus atom, a carbon atom, or a silicon atom, and in a case where X¹ represents an oxygen atom, a is 0, in a case where X¹ represents a nitrogen atom or a phosphorus atom, a is 1, and in a case where X¹ represents a carbon atom or a silicon atom, a is 2,
Y¹ and Y² each represent a benzene ring or a naphthalene ring, and in a case where Y¹ and/or Y² represents a benzene ring, b or c corresponding to Y¹ and/or Y² representing the benzene ring is 4, and in a case where Y¹ and/or Y² represents a naphthalene ring, b and/or c corresponding to Y¹ and/or Y² representing the naphthalene ring is 6,
R¹ to R³ each represent hydrogen or a substituent represented by *—Z¹(R⁴)_{d} (in which * represents a binding site), and in a case where a plurality of R¹s, a plurality of R²s, and a plurality of R³s are present, the plurality of R¹s, the plurality of R²s, and the plurality of R³s may be of an identical kind or of different kinds, and all of the plurality of R¹s, the plurality of R²s, and the plurality of R³s in the general formula (1) are not simultaneously hydrogen,
Z¹ represents a single bond, a divalent or higher saturated hydrocarbon group, or a divalent or higher unsaturated hydrocarbon group, and the divalent or higher saturated hydrocarbon group or the divalent or higher unsaturated hydrocarbon group may contain an ether bond and/or a thioether bond, and in a case where Z¹ represents a single bond, d is 1, and in a case where Z¹ represents a divalent or higher saturated hydrocarbon group or a divalent or higher unsaturated hydrocarbon group, d is an integer of 1 or more,
R⁴ represents hydrogen or a polymerizable substituent, and in a case where a plurality of R⁴s is present, the plurality of R⁴s may be of an identical kind or of different kinds, and all of the plurality of R⁴s in the general formula (1) is not simultaneously hydrogen.

2. The hologram recording medium according to claim 1, wherein
the photosensitive layer further contains a photopolymerizable initiator, and
the photopolymerizable initiator contains at least one of an organic boron salt-based initiator or an onium salt-based initiator.

3. The hologram recording medium according to claim 1, wherein
the photosensitive layer further contains a photopolymerizable initiator, and
the photopolymerization initiator contains an organic boron salt-based initiator and an onium salt-based initiator.

4. The hologram recording medium according to claim 1, wherein the compound is represented by a general formula (1-1) described below: 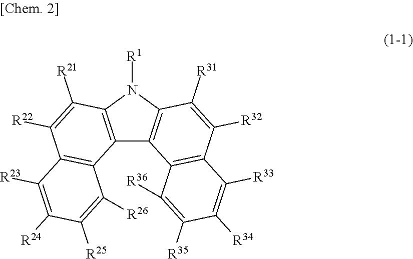
wherein, R¹, R²¹ to R²⁶, and R³¹ to R³⁶ represent hydrogen or a substituent represented by *—Z¹(R⁴)_{d} (in which * represents a binding site), R¹, R²¹ to R²⁶, and R³¹ to R³⁶ may be of an identical kind or of different kinds, and all of R¹, R²¹ to R²⁶, and R³¹ to R³⁶ in the general formula (1-1) are not simultaneously hydrogen,
Z¹ represents a single bond, a divalent or higher saturated hydrocarbon group, or a divalent or higher unsaturated hydrocarbon group, and the divalent or higher saturated hydrocarbon group or the divalent or higher unsaturated hydrocarbon group may contain an ether bond and/or a thioether bond, and in a case where Z¹ represents a single bond, d is 1, and in a case where Z¹ represents a divalent or higher saturated hydrocarbon group or a divalent or higher unsaturated hydrocarbon group, d is an integer of 1 or more,
R⁴ represents hydrogen or a polymerizable substituent, and in a case where a plurality of R⁴s is present, the plurality of R⁴s may be of an identical kind or of different kinds, and all of the plurality of R⁴s in the general formula (1-1) is not simultaneously hydrogen.

5. The hologram recording medium according to claim 1, wherein the compound is represented by a general formula (1-2) described below: 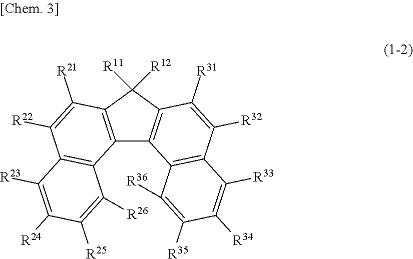
wherein, R¹¹, R¹², R²¹ to R²⁶, and R³¹ to R³⁶ represent hydrogen or a substituent represented by *—Z¹(R⁴)_{d} (in which * represents a binding site), R¹¹, R¹², R²¹ to R²⁶, and R³¹ to R³⁶ may be of an identical kind or of different kinds, and all of R¹¹, R¹², R²¹ to R²⁶, and R³¹ to R³⁶ in the general formula (1-2) are not simultaneously hydrogen,
Z¹ represents a single bond, a divalent or higher saturated hydrocarbon group, or a divalent or higher unsaturated hydrocarbon group, and the divalent or higher saturated hydrocarbon group or the divalent or higher unsaturated hydrocarbon group may contain an ether bond and/or a thioether bond, and in a case where Z¹ represents a single bond, d is 1, and in a case where Z¹ represents a divalent or higher saturated hydrocarbon group or a divalent or higher unsaturated hydrocarbon group, d is an integer of 1 or more,
R⁴ represents hydrogen or a polymerizable substituent, and in a case where a plurality of R⁴s is present, the plurality of R⁴s may be of an identical kind or of different kinds, and all of the plurality of R⁴s in the general formula (1-2) is not simultaneously hydrogen.

6. The hologram recording medium according to claim 1, wherein the photosensitive layer further contains a binder resin.

7. The hologram recording medium according to claim 1, wherein Y¹ and Y² in the general formula (1) are not simultaneously a benzene ring.

8. The hologram recording medium according to claim 1, wherein the oxygen permeability of the base material measured in the environment at 23° C. and 0% RH is more than 5 cm³·(m²·day·atm)⁻¹ and 10000 cm³·(m²·day·atm)⁻¹ or less.

9. A hologram optical element comprising the hologram recording medium according to claim 1, wherein
the polymerizable compound is polymerized.

10. An optical device comprising the hologram optical element according to claim 9.

11. An optical component comprising the hologram optical element according to claim 9.

12. A method for forming a hologram diffraction grating, the method comprising
selectively reacting a hologram recording medium including a base material and a photosensitive layer, by using an electromagnetic ray having an amplitude modulated spatially,
the base material having an oxygen permeability of more than 0.1 cm³·(m²·day·atm)⁻¹ and 10000 cm³·(m²·day·atm)⁻¹ or less, the oxygen permeability measured in an environment at 23° C. and 0% RH,
the photosensitive layer containing a polymerizable compound, the polymerizable compound represented by a general formula (1) described below: 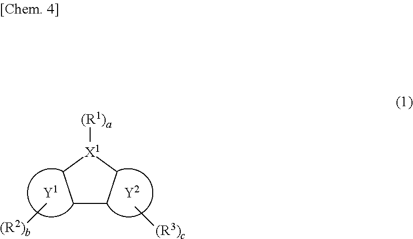
wherein X¹ represents an oxygen atom, a nitrogen atom, a phosphorus atom, a carbon atom, or a silicon atom, and in a case where X¹ represents an oxygen atom, a is 0, in a case where X¹ represents a nitrogen atom or a phosphorus atom, a is 1, and in a case where X¹ represents a carbon atom or a silicon atom, a is 2,
Y¹ and Y² each represent a benzene ring or a naphthalene ring, and in a case where Y¹ and/or Y² represents a benzene ring, b or c corresponding to Y¹ and/or Y² representing the benzene ring is 4, and in a case where Y¹ and/or Y² represents a naphthalene ring, b and/or c corresponding to Y¹ and/or Y² representing the naphthalene ring is 6,
R¹ to R³ each represent hydrogen or a substituent represented by *—Z¹(R⁴)_{d} (in which * represents a binding site), and in a case where a plurality of R¹s, a plurality of R²s, and a plurality of R³s are present, the plurality of R¹s, the plurality of R²s, and the plurality of R³s may be of an identical kind or of different kinds, and all of the plurality of R¹s, the plurality of R²s, and the plurality of R³s in the general formula (1) are not simultaneously hydrogen,
Z¹ represents a single bond, a divalent or higher saturated hydrocarbon group, or a divalent or higher unsaturated hydrocarbon group, and the divalent or higher saturated hydrocarbon group or the divalent or higher unsaturated hydrocarbon group may contain an ether bond and/or a thioether bond, and in a case where Z¹ represents a single bond, d is 1, and in a case where Z¹ represents a divalent or higher saturated hydrocarbon group or a divalent or higher unsaturated hydrocarbon group, d is an integer of 1 or more,
R⁴ represents hydrogen or a polymerizable substituent, and in a case where a plurality of R⁴s is present, the plurality of R⁴s may be of an identical kind or of different kinds, and all of the plurality of R⁴s in the general formula (1) is not simultaneously hydrogen.
